(19) United States
(12) Patent Application Publication
(10) Pub. No.: US 2025/0312350 A1
(43) Pub. Date: Oct. 9, 2025

(54) INHIBITORS OF RLIP76

(71) Applicant: AVESTA76 THERAPEUTICS, INC., Los Angeles, CA (US)

(72) Inventors: Sanjay AWASTHI, Los Angeles, CA (US); Alexander BRIDGES, Los Angeles, CA (US); Jennifer MURPHY, Los Angeles, CA (US)

(73) Assignee: AVESTA76 THERAPEUTICS, INC., Los Angeles, CA (US)

(21) Appl. No.: 18/866,828

(22) PCT Filed: May 17, 2023
(86) PCT No.: PCT/US2023/067123

§ 371 Date: Nov. 18, 2024

Related U.S. Application Data
(60) Provisional application No. 63/342,985, filed on May 17, 2022.

(51) Int. Cl. A61K 31/5377; A61K 31/4985; A61K 45/06; A61P 35/00

(52) U.S. Cl. CPC A61K 31/5377; A61K 31/4985; A61K 45/06; A61P 35/00

(57) ABSTRACT

Methods and compositions involving inhibitors of RLIP76 for the treatment or prevention of various types of cancer are disclosed.

This application claims priority of U.S. Provisional Patent Application No. 63/342,985, filed May 17, 2022, which is hereby incorporated by reference in its entirety.

### BACKGROUND OF THE INVENTION

### 1. Field of the Invention

The present invention relates generally to the fields of biochemistry, cell biology, organic chemistry, and oncology.

### 2. Description of Related Art

RLIP76, a stress-responsive, multi-functional protein with multi-specific transport activity towards glutathione-conjugates (GS-E) and chemotherapeutic agents, is frequently over-expressed in malignant cells. RLIP76 has also been shown to be an effective transporter of many conventional chemotherapeutic drugs. Such transport, if inhibited, can lead to increased cellular accumulation of drugs which in turn translates to enhanced drug sensitivity.

The expression of RLIP76 is significantly greater in human cancer cells of diverse origin as compared to the non-malignant cells. The primary focus on inhibiting RLIP76 to treat cancer has been on using biologics. For example, antibodies have been used for targeting cell surface epitopes. siRNAs and anti-sense phosphorothioate oligonucleotides have been used for depleting RLIP76 levels in cells, which can result in apoptosis in malignant cells. Administration of RLIP76 antibodies, siRNA, or anti-sense oligonucleotides to mice bearing syngeneic B16 mouse melanoma tumors has been shown to cause rapid and complete regression of tumors. While attempts have been made to develop small molecules to target RLIP76, to date these attempts have largely been unsuccessful.

### SUMMARY OF THE INVENTION

A solution to at least one or more of the aforementioned problems has been identified. In one aspect of the present disclosure, a solution includes identification and characterization of small molecule compounds for the treatment or prevention of cancer via targeting RLIP76 activity in cancer cells. By way of example, and in the context of the present disclosure, the following compounds have been found to be effective at targeting RLIP76 activity and can be used to treat or prevent cancer in subjects:

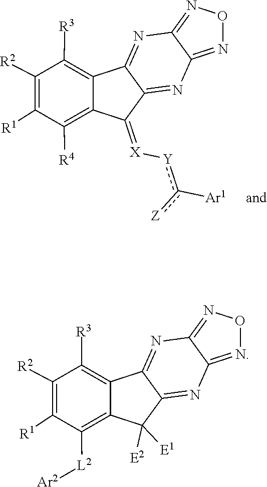

Also disclosed are methods for cancer treatment, methods for cancer prevention, methods for stopping or reducing tumor growth, methods for killing cancer cells, methods for reducing oncogenesis, and methods for inhibiting RAL-interacting proteins. Methods of the present disclosure can include at least 1, 2, 3, 4, 5, or more of the following steps: administering a therapeutic composition comprising a compound of formula (I) and/or a compound of formula (II) to a subject, providing a RAL-interacting protein inhibitor to a cell, administering an additional cancer therapy to a subject, monitoring the effectiveness of a treatment provided to a subject, and altering a dose of a therapeutic composition administered to an individual. Any one or more of the preceeding steps, or any step disclosed herein, may be excluded from certain aspects of the disclosure.

In one aspect of the present disclosure, there is disclosed a method of treating or preventing cancer in a subject, the method comprising administering to the subject a compound of formula (I):

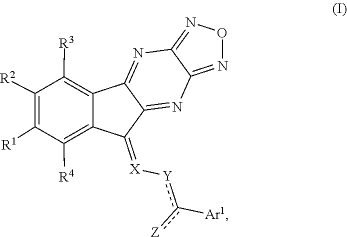

wherein X is N or CH;
Y is NH or O when Z is a carbonyl oxygen, or CH when Z is F;
Z is ═O or F;
R¹ is OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, a substituted or unsubstituted heterocycloalkoxy, or a substituted or unsubstituted heterocyclosulfide;
Ar¹ is

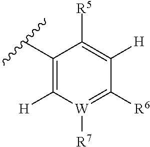

W is N or C;
R² and R⁴ are each independently H, halogen, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, (C₄₋₇ heterocycloalkyl-C₀₋₄ alkyl-L¹)ₚ, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ;
R³ is H, halogen, or C₁₋₆ lower alkyl;
R⁵ is NH₂, OH or F;
R⁶ is H, OH, F, Cl, Br, CH₃, ethynyl, cyano, a substituted or unsubstituted alkenyl, a substituted or unsubstituted alkoxy, or a substituted or unsubstituted sulfide;
R⁷ is nothing when W is N, or H, OH, OR⁹, OCOR¹¹, OCOR⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ when W is C;
R⁹ and R¹⁰ are each independently H, R³, or R⁹ and R¹⁰, and the N atom that connects R⁹ and R¹⁰ can form a heterocyclic ring of 4 to 8 members which can further comprise N, O, S, or SO₂;
R¹¹ is C₁₋₆ lower alkyl, C₂₋₆ lower alkenyl, C₂₋₆ lower alkynyl, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
L¹ is independently at each occurrence a bond, O, NH, NR⁴, N(C₁₋₆ lower acyl), S, SO, SO₂, CO, CONR⁴, CO₂, NR⁴CO, OCONH, OCONR⁴, OCO₂, NR⁴CONR⁴, NR⁴CSNR⁴, NR⁴CO, OCO, SO₂NR⁴, or NR⁴SO₂;
p is 1-3, wherein each repeat unit can be the same or different with respect to both cyclic and linear elements; and wherein each of R¹—R¹¹, independently, can optionally be substituted with up to 3 substituents selected from the group consisting of OH, CN, C₁₋₆ lower alkyl, C₁₋₆ lower alkoxy, C₁₋₆ lower alkylthio, C₁₋₆ lower alkylamino, C₁₋₆ lower dialkylamino, C₁₋₆ lower acyloxy, optionally substituted phenyl, optionally substituted C₅₋₆ heteroaryl, halogen, CF₃, OCF₃, oxo, (OCH₂CH₂O)₁₋₁₀OH, and (OCH₂CH₂O)₁₋₁₀OCH₃; or an enantiomer, diastereoisomer, racemic mixture, or salt(s) thereof. It is specifically contemplated that one or more of the substituents listed as being present in certain aspects may be excluded in other aspects. For example, it is specifically contemplated that R¹ is not OH in certain aspects. In some aspects, only one of R¹, R², R³, R⁴ are not hydrogen. In some aspects, R¹, R², R³, R⁴ are all hydrogen. In some aspects, any hydrogen or carbon present in the compound are replaced by a corresponding isotope ²H, ³H, ¹³C or ¹⁴C. In some aspects, the compound of formula (I) is further defined as:

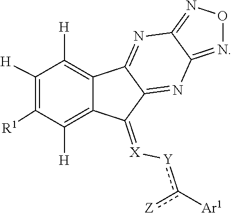

In some aspects, a method of treating or preventing cancer in a subject comprises administering to the subject at least one of the following compounds of formula (I):

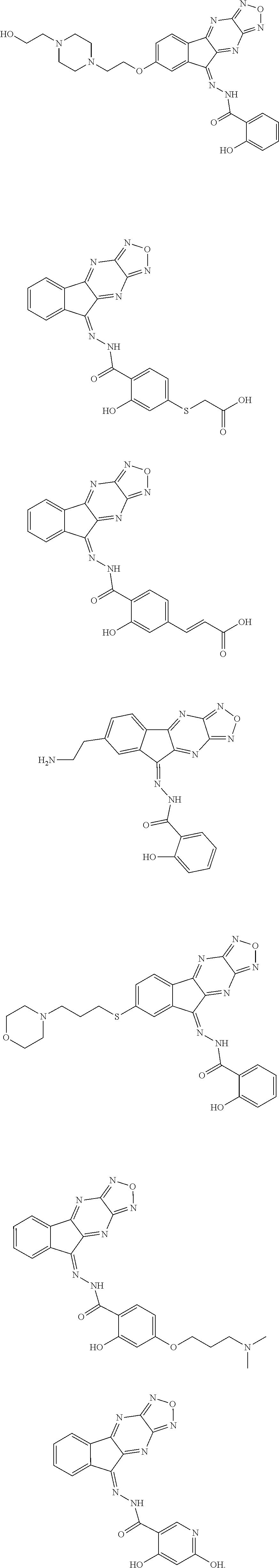

In some aspects, the compound of formula (I) inhibits a RAL-interacting protein. In some aspects, the RAL-interacting protein is RLIP76. In some aspects, the compound binds to transporter substrate binding site of RLIP76. In some aspects, administration of the compound of formula (I) overcomes deleterious effects of p53 gene loss. In some aspects, the subject has cancer, is suspected of having, or is diagnosed with cancer. In some aspects, the cancer that is treated or prevented by administration of a compound of formula (I) is, or is not, breast cancer, colon cancer, lung cancer, hepatocellular cancer, pancreatic cancer, prostate cancer, glioblastoma, melanoma, or ovarian cancer. In some aspects, the breast cancer is triple-negative breast cancer, ductal carcinoma in situ, invasive ductal carcinoma, tubular carcinoma of a breast, medullary carcinoma of a breast, mucinous carcinoma of a breast, papillary carcinoma of a breast, cribriform carcinoma of a breast, invasive lobular carcinoma, inflammatory breast cancer, lobular carcinoma in situ, male breast cancer, molecular subtypes of breast cancer, Paget's disease of a nipple, phyllodes tumors of a breast, metastatic breast cancer, or combinations thereof. In some aspects, the compound of formula (I) is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof. In some aspects, administration of the compound of formula (I) is done prior to, concurrently with, or subsequent to chemotherapy, surgical treatment, or radiation treatment. In some aspects, the compound of formula (I) is administered to the subject at least two, three, four, five, six, seven, eight, nine or ten times. In some aspects, the subject is administered at least, about, at most, or at 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, 100, 150, 200, 250, or 300 μg/kg or mg/kg of the compound of formula (I). In some aspects, subject is further administered a distinct cancer therapy. In some aspects, the distinct cancer therapy comprises surgery, radiotherapy, chemotherapy, toxin therapy, immunotherapy, cryotherapy or gene therapy. In some aspects, the compound of formula (I) is not:

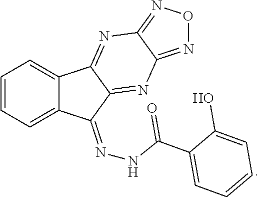 [SMILES: O=C(N/N=C1/c2ccccc2-c2nc3nonc3nc21)c1ccccc1O]

Some aspects of the disclosure are directed to a method for inhibiting an RAL-interacting protein in a cell comprising providing to the cell an effective amount of an RAL-interacting protein inhibitor, wherein the RAL-interacting protein inhibitor is a compound of the formula (I):

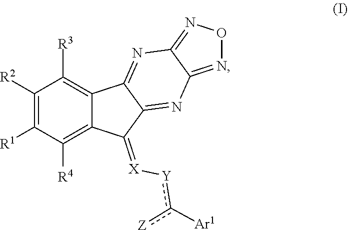

wherein X is N or CH;
Y is NH or O when Z is a carbonyl oxygen, or CH when Z is F;
Z is ═O or F;
R¹ is OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, a substituted or unsubstituted heterocycloalkoxy, or a substituted or unsubstituted heterocyclosulfide;
Ar¹ is

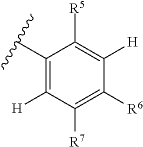

W is N or C;
R² and R⁴ are each independently H, halogen, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₈₋₁₀ bridged bicycloalkyl, C₆₋₁₀ bridged bicycloheteroalkyl, (C₄₋₇ heterocycloalkyl-C₀₋₄ alkyl-L¹)ₚ, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, or a substituted or unsubstituted heterocycloakyl;
R³ is H, halogen, or C₁₋₆ lower alkyl;
R⁵ is NH₂, OH or F;
R⁶ is H, OH, F, Cl, Br, CH₃, ethynyl, cyano, a substituted or unsubstituted alkenyl, a substituted or unsubstituted alkoxy, or a substituted or unsubstituted sulfide
R⁷ is nothing when W is N, or H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ when W is C;
R⁹ and R¹⁰ are each independently H, R³, or R⁹ and R¹⁰, and the N atom that connects R⁹ and R¹⁰ can form a heterocyclic ring of 4 to 8 members which can further comprise N, O, S, or SO₂;
R¹¹ is C₁₋₆ lower alkyl, C₂₋₆ lower alkenyl, C₂₋₆ lower alkynyl, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
L¹ is independently at each occurrence a bond, O, NH, NR⁴, N(C₁₋₆ lower acyl), S, SO, SO₂, CO, CONR⁴, CO₂, NR⁴CO, OCONH, OCONR⁴, OCO₂, NR⁴CONR⁴, NR⁴CSNR⁴, NR⁴CO, OCO, SO₂NR⁴, or NR⁴SO₂;
p is 1-3, wherein each repeat unit can be the same or different with respect to both cyclic and linear elements; and wherein each of R¹—R¹¹, independently, can optionally be substituted with up to 3 substituents selected from the group consisting of OH, CN, C₁₋₆ lower alkyl, C₁₋₆ lower alkoxy, C₁₋₆ lower alkylthio, C₁₋₆ lower alkylamino, C₁₋₆ lower dialkylamino, C₁₋₆ lower acyloxy, optionally substituted phenyl, optionally substituted C₅₋₆ heteroaryl, halogen, CF₃, OCF₃, oxo, (OCH₂CH₂O)₁₋₁₀OH, and (OCH₂CH₂O)₁₋₁₀OCH₃; or an enantiomer, diastereoisomer, racemic mixture, or salt(s) thereof or salt(s) thereof. It is specifically contemplated that one or more of the substituents listed as being present in certain aspects may be excluded in other aspects. For example, it is specifically contemplated that R¹ is not OH in certain aspects. In some aspects, only one of R¹, R², R³, R⁴ are not hydrogen. In some aspects, R¹, R², R³, R⁴ are all hydrogen. In some aspects, any hydrogen or carbon present in the compound are replaced by a corresponding isotope ²H, ³H, ¹³C or ¹⁴C. In some aspects, the compound of formula (I) is further defined as:

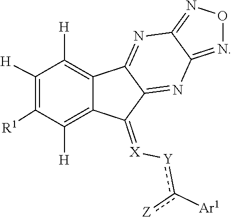

In some aspects, the RAL-interacting protein inhibitor is at least one of the following compounds of formula (I):

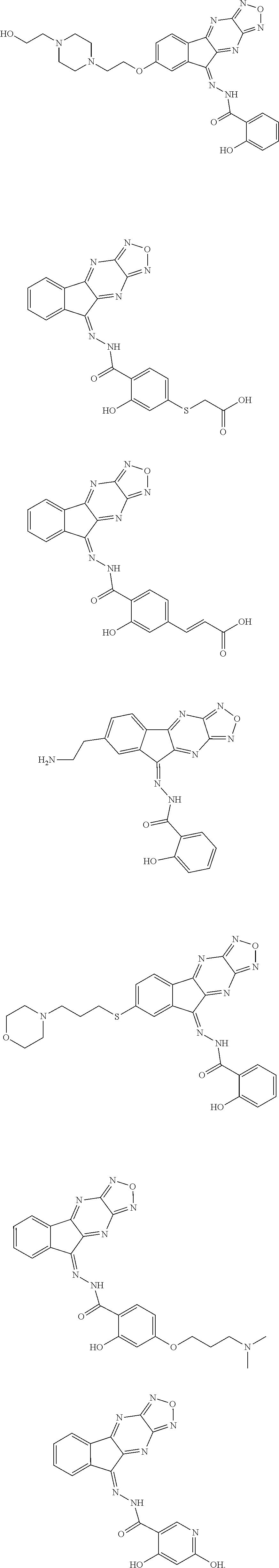

In some aspects, the cell is a cancer cell. In some aspects, the cell is in a patient. In some aspects, the patient is a cancer patient. In some aspects, the cancer patient has breast cancer, colon cancer, lung cancer, hepatocellular cancer, pancreatic cancer, prostate cancer, glioblastoma, melanoma, or ovarian cancer. In some aspects, the RAL-interacting protein is RLIP76. In some aspects, the RAL-interacting protein inhibitor binds to a transporter substrate binding site of RLIP76. In some aspects, the RAL-interacting protein inhibitor is provided to the cell multiple times. In some aspects, the RAL-interacting protein inhibitor is not

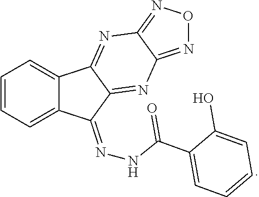 [SMILES: O=C(N/N=C1/c2ccccc2-c2nc3nonc3nc21)c1ccccc1O]

Some aspects of the present disclosure are directed to a method of treating or preventing cancer in a subject, comprising administering to the subject a compound of formula (II):

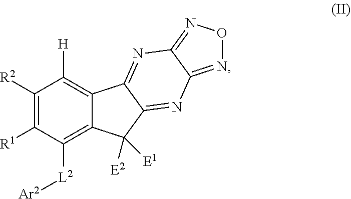

E¹ and E² are independently H or F, or one of E¹ and E² is H and the other OH, CCH or CN, or E¹ and E² may be taken together to be ═O or ═CH₂;
Ar² is

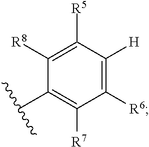

L₂ is —CH₂CH₂—, —CH₂CHR²—, —CH₂NH—, —NHCH₂—, —CH₂NR³—, —NHCHR³—, —CH₂O—, —OCH₂—, —OCHR³—, —COCH₂—, —COCHR²—, —CH₂CO—, —CH₂S—, —CH₂SO—, —CH₂SO(NH)—, —SCH₂—, —SOCH₂—, —SO₂CH₂—, —SO(NH)CH₂—, —SCHR³—, —SOCHR³—, —SO₂CHR³—, —SO(NH)CHR³—, —CH₂SO₂—, —SO₂CHR²—, —SOCHR²—, —SONHCHR²—, —NHS—, —NHSO—, —NHSO₂—, —SNH—, —SONH—, —SO₂NH—, —NHNH—, —SNR³—, —SONR³—, —SO₂NR³—, —NHR³—, —R³NH—, —NHO—, —ONH—, CONH—, —NHCO—, —CONH—, —ONR³—, —NR³CO—, —CONR³—, —OCO— or —COO—;
R¹ is OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, a substituted or unsubstituted heterocycloalkoxy, or a substituted or unsubstituted heterocyclosulfide;
R² is H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
R³ is C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, with the proviso that the L¹ directly bonded to L² is only selected from a bond, CO, CONR⁴ or CO₂;
R⁴ is H or C₁₋₆ lower alkyl;
R⁵ is NH₂, OH or F;
R⁶ is H, F, Cl, Br, CH₃, ethynyl or cyano;
R⁷ is H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
R⁸ is CH₂OH, CH₂F, CHF₂, CF₃, CHO, CO₂H, CO₂Me, CONH₂, or CONHMe;
R⁹ and R¹⁰ are each independently H, R³, or R⁹ and R¹⁰, and the N atom that connects R⁹ and R¹⁰ can form a heterocyclic ring of 4 to 8 members which can further comprise N, O, S, or SO₂;
R¹¹ is C₁₋₆ lower alkyl, C₂₋₆ lower alkenyl, C₂₋₆ lower alkynyl, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
L¹ is independently at each occurrence a bond, O, NH, NR⁴, N(C₁₋₆ lower acyl), S, SO, SO₂, CO, CONR⁴, CO₂, NR⁴CO, OCONH, OCONR⁴, OCO₂, NR⁴CONR⁴, NR⁴CSNR⁴, NR⁴CO, OCO, SO₂NR⁴, or NR⁴SO;
p is 1-3, wherein each repeat unit can be the same or different with respect to both cyclic and linear elements; wherein each of R¹—R¹¹ can be optionally, independently be substituted with up to 3 substituents from the following list, OH, CN, C₁₋₆ lower alkyl, C₁₋₆ lower alkoxy, C₁₋₆ lower alkylthio, C₁₋₆ lower alkylamino, C₁₋₆ lower dialkylamino, C₁₋₆ lower acyloxy, optionally substituted phenyl, optionally substituted C₅₋₆ heteroaryl, halogen, CF₃, CF₃O, oxo, (OCH₂CH₂O)₁₋₁₀OH, and (OCH₂CH₂O)₁₋₁₀OCH₃; or an enantiomer, diastereoisomer, racemic mixture, or salt(s) thereof or salt(s) thereof. It is specifically contemplated that one or more of the substituents listed as being present in certain aspects may be excluded in other aspects. For example, it is specifically contemplated that R¹ is not OH in certain aspects. In some aspects, only one of R¹, R², R³, R⁴ are not hydrogen. In some aspects, R¹, R², R³, R⁴ are all hydrogen. In some aspects, any hydrogen or carbon present in the compound are replaced by a corresponding isotope ²H, ³H, ¹³C or ¹⁴C. In some aspects the compound of formula (II) is further defined as:

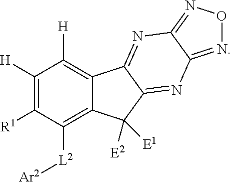

In some aspects, a method of treating or preventing cancer in a subject comprises administering to the subject at least one of the following compounds of formula (II), or excludes administering to the subject at least one of the following compounds of formula (II):

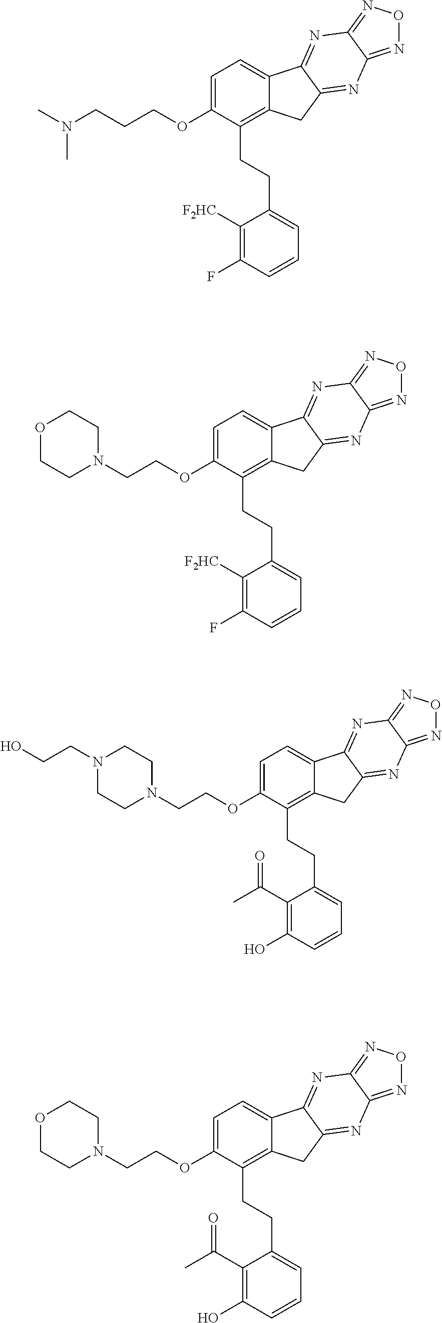 [SMILES: CN(C)CCCOc1ccc2c(c1CCc1cccc(F)c1C(F)F)Cc1nc3nonc3nc1-2 ; Fc1cccc(CCc2c(OCCN3CCOCC3)ccc3c2Cc2nc4nonc4nc2-3)c1C(F)F ; CC(=O)c1c(O)cccc1CCc1c(OCCN2CCN(CCO)CC2)ccc2c1Cc1nc3nonc3nc1-2 ; CC(=O)c1c(O)cccc1CCc1c(OCCN2CCOCC2)ccc2c1Cc1nc3nonc3nc1-2]

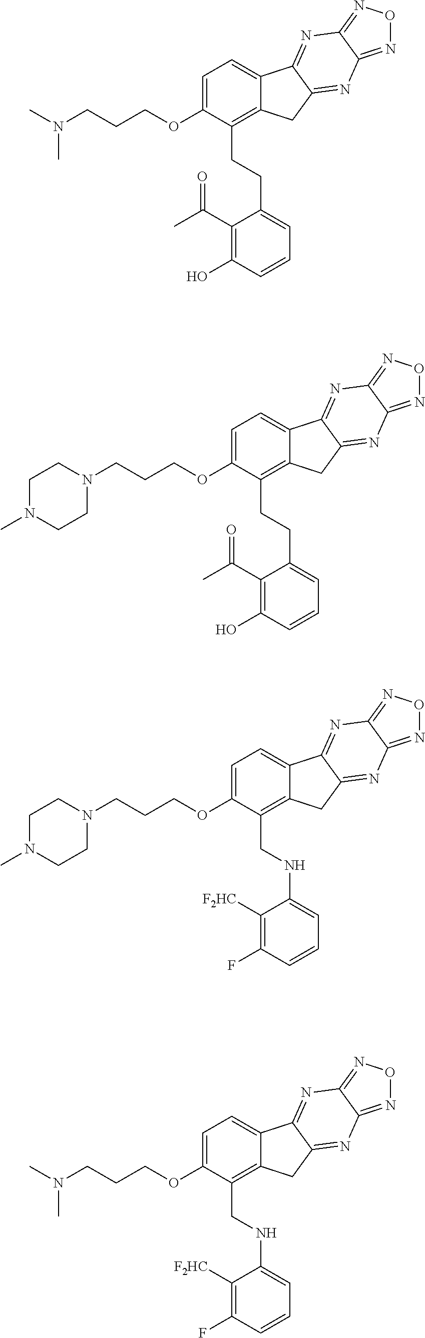 [SMILES: CC(=O)c1c(O)cccc1CCc1c(OCCCN(C)C)ccc2c1Cc1nc3nonc3nc1-2 ; CC(=O)c1c(O)cccc1CCc1c(OCCCN2CCN(C)CC2)ccc2c1Cc1nc3nonc3nc1-2 ; CN1CCN(CCCOc2ccc3c(c2CNc2cccc(F)c2C(F)F)Cc2nc4nonc4nc2-3)CC1 ; CN(C)CCCOc1ccc2c(c1CNc1cccc(F)c1C(F)F)Cc1nc3nonc3nc1-2]

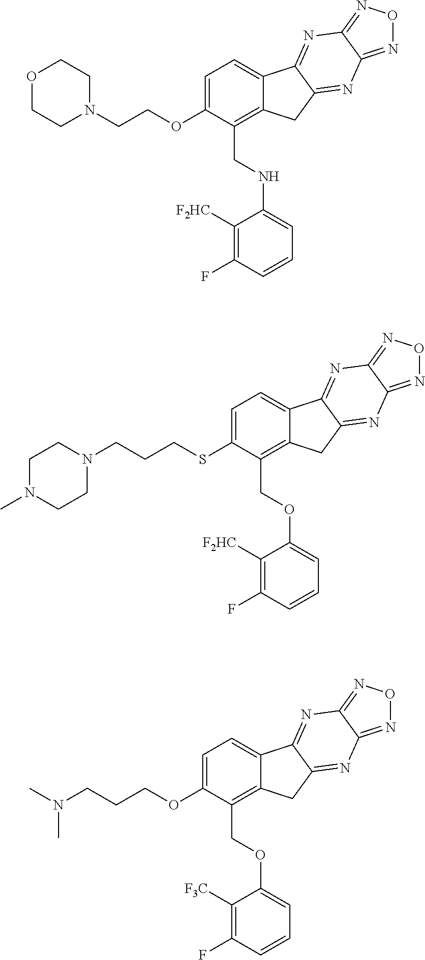 [SMILES: Fc1cccc(NCc2c(OCCN3CCOCC3)ccc3c2Cc2nc4nonc4nc2-3)c1C(F)F ; CN1CCN(CCCSc2ccc3c(c2COc2cccc(F)c2C(F)F)Cc2nc4nonc4nc2-3)CC1 ; CN(C)CCCOc1ccc2c(c1COc1cccc(F)c1C(F)(F)F)Cc1nc3nonc3nc1-2]

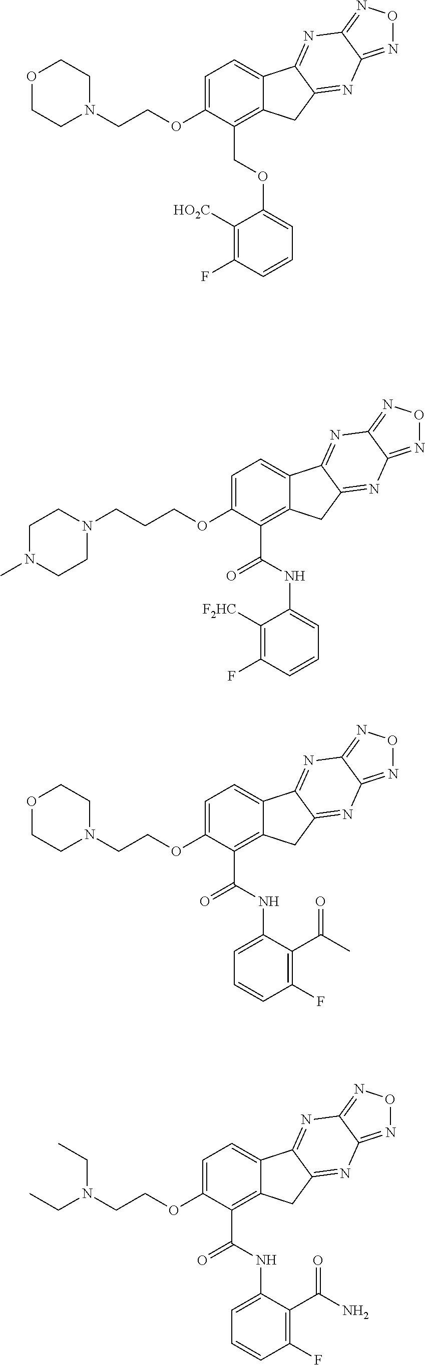 [SMILES: O=C(O)c1c(F)cccc1OCc1c(OCCN2CCOCC2)ccc2c1Cc1nc3nonc3nc1-2 ; CN1CCN(CCCOc2ccc3c(c2C(=O)Nc2cccc(F)c2C(F)F)Cc2nc4nonc4nc2-3)CC1 ; CC(=O)c1c(F)cccc1NC(=O)c1c(OCCN2CCOCC2)ccc2c1Cc1nc3nonc3nc1-2 ; CCN(CC)CCOc1ccc2c(c1C(=O)Nc1cccc(F)c1C(N)=O)Cc1nc3nonc3nc1-2]

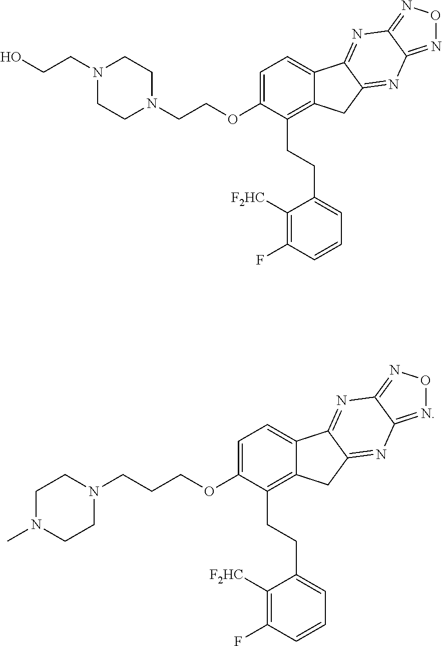 [SMILES: OCCN1CCN(CCOc2ccc3c(c2CCc2cccc(F)c2C(F)F)Cc2nc4nonc4nc2-3)CC1 ; CN1CCN(CCCOc2ccc3c(c2CCc2cccc(F)c2C(F)F)Cc2nc4nonc4nc2-3)CC1]

In some aspects, the compound of formula (II) inhibits an RAL-interacting protein. In some aspects, the RAL-interacting protein is RLIP76. In some aspects, the compound binds to a transporter substrate binding site of RLIP76. In some aspects, administration of the compound of formula (II) overcomes deleterious effects of p53 gene loss. In some aspects, the subject has cancer, is suspected of having, or is diagnosed with cancer. In some aspects, the cancer that is treated or prevented by administration of a compound of formula (II) is, or is not, breast cancer, colon cancer, lung cancer, hepatocellular cancer, pancreatic cancer, prostate cancer, glioblastoma, melanoma, or ovarian cancer. In some aspects, the breast cancer is triple-negative breast cancer, ductal carcinoma in situ, invasive ductal carcinoma, tubular carcinoma of a breast, medullary carcinoma of a breast, mucinous carcinoma of a breast, papillary carcinoma of a breast, cribriform carcinoma of a breast, invasive lobular carcinoma, inflammatory breast cancer, lobular carcinoma in situ, male breast cancer, molecular subtypes of breast cancer, Paget's disease of a nipple, phyllodes tumors of a breast, metastatic breast cancer, or combinations thereof. In some aspects, the compound of formula (II) is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof. In some aspects, administration of the compound of formula (II) is done prior to, concurrently with, or subsequent to chemotherapy, surgical treatment, or radiation treatment. In some aspects, the compound of formula (II) is administered to the subject at least, at most, about, or at two, three, four, five, six, seven, eight, nine or ten times. In some aspects, the subject is administered at least, at most, about, or at 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, 100, 150, 200, 250, or 300 μg/kg or mg/kg of the compound of formula (11) or any range derivable therein. In some aspects, subject is further administered a distinct cancer therapy. In some aspects, the distinct cancer therapy comprises surgery, radiotherapy, chemotherapy, toxin therapy, immunotherapy, cryotherapy or gene therapy. In some aspects, no distinct cancer therapy is administered.

Some aspects of the present disclosure are directed to a method for inhibiting an RAL-interacting protein in a cell comprising providing to the cell an effective amount of an RAL-interacting protein inhibitor, wherein the RAL-interacting protein inhibitor is a compound of the formula (11):

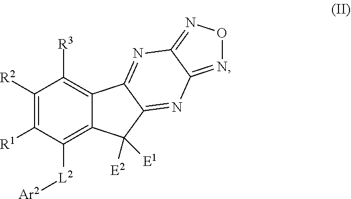

E¹ and E² are independently H or F, or one of E¹ and E² is H and the other OH, CCH or CN, or E¹ and E² may be taken together to be ═O or ═CH₂;
Ar² is

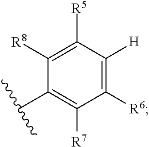

L₂ is —CH₂CH₂—, —CH₂CHR²—, —CH₂NH—, —NHCH₂—, —CH₂NR³—, —NHCHR³—, —CH₂O—, —OCH₂—, —OCHR³—, —COCH₂—, —COCHR²—, —CH₂CO—, —CH₂S—, —CH₂SO—, —CH₂SO(NH)—, —SCH₂—, —SOCH₂—, —SO₂CH₂—, —SO(NH)CH₂—, —SCHR³—, —SOCHR³—, —SO₂CHR³—, —SO(NH)CHR³—, —CH₂SO₂—, —SO₂CHR²—, —SOCHR²—, —SONHCHR²—, —NHS—, —NHSO—, —NHSO₂—, —SNH—, —SONH—, —SO₂NH—, —NHNH—, —SNR³—, —SONR³—, —SO₂NR³—, —NHR³—, —R³NH—, —NHO—, —ONH—, CONH—, —NHCO—, —CONH—, —ONR³—, —NR³CO—, —CONR³—, —OCO— or —COO—;
R¹ is OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, a substituted or unsubstituted heterocycloalkoxy, or a substituted or unsubstituted heterocyclosulfide;
R² is H, OH, OR⁹, OCOR¹¹, OCOR⁹, OCONHR⁹, OCONR⁹R¹⁰, NH₂ NHR⁹, NR⁹R¹⁰, NHCOR¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
R³ is C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, with the proviso that the L¹ directly bonded to L² is only selected from a bond, CO, CONR⁴ or CO₂;
R⁴ is H or C₁₋₆ lower alkyl;
R⁵ is NH₂, OH or F;
R⁶ is H, F, Cl, Br, CH₃, ethynyl or cyano;
R⁷ is H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
R⁸ is CH₂OH, CH₂F, CHF₂, CF₃, CHO, CO₂H, CO₂Me, CONH₂, or CONHMe;
R⁹ and R¹⁰ are each independently H, R³, or R⁹ and R¹⁰, and the N atom that connects R⁹ and R¹⁰ can form a heterocyclic ring of 4 to 8 members which can further comprise N, O, S, or SO₂;
R¹¹ is C₁₋₆ lower alkyl, C₂₋₆ lower alkenyl, C₂₋₆ lower alkynyl, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ; L¹ is independently at each occurrence a bond, O, NH, NR⁴, N(C₁₋₆ lower acyl), S, SO, SO₂, CO, CONR⁴, CO₂, NR⁴CO, OCONH, OCONR⁴, OCO₂, NR⁴CONR⁴, NR⁴CSNR⁴, NR⁴CO, OCO, SO₂NR⁴, or NR⁴SO;
p is 1-3, wherein each repeat unit can be the same or different with respect to both cyclic and linear elements; wherein each of R¹—R¹¹, independently, can optionally be substituted with up to 3 substituents from the following list, OH, CN, C₁₋₆ lower alkyl, C₁₋₆ lower alkoxy, C₁₋₆ lower alkylthio, C₁₋₆ lower alkylamino, C₁₋₆ lower dialkylamino, C₁₋₆ lower acyloxy, optionally substituted phenyl, optionally substituted C₅₋₆ heteroaryl, halogen, CF₃, CF₃O, oxo, (OCH₂CH₂O)₁₋₁₀OH, and (OCH₂CH₂O)₁₋₁₀OCH₃; or an enantiomer, diastereoisomer, racemic mixture, or salt(s) thereof or salt(s) thereof. It is specifically contemplated that one or more of the substituents listed as being present in certain aspects may be excluded in other aspects. For example, it is specifically contemplated that R¹ is not OH in certain aspects. In some aspects, only one of R¹, R², R³, R⁴ are not hydrogen. In some aspects, R¹, R², R³, R⁴ are all hydrogen. In some aspects, any hydrogen or carbon present in the compound are replaced by a corresponding isotope ²H, ³H, ¹³C or ¹⁴C. In some aspects the compound of formula (II) is further defined as:

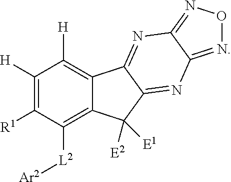

In some aspects, the RAL-interacting protein inhibitor is at least one of the following compounds of formula (II) or is not at least one of the following compounds of formula (II):

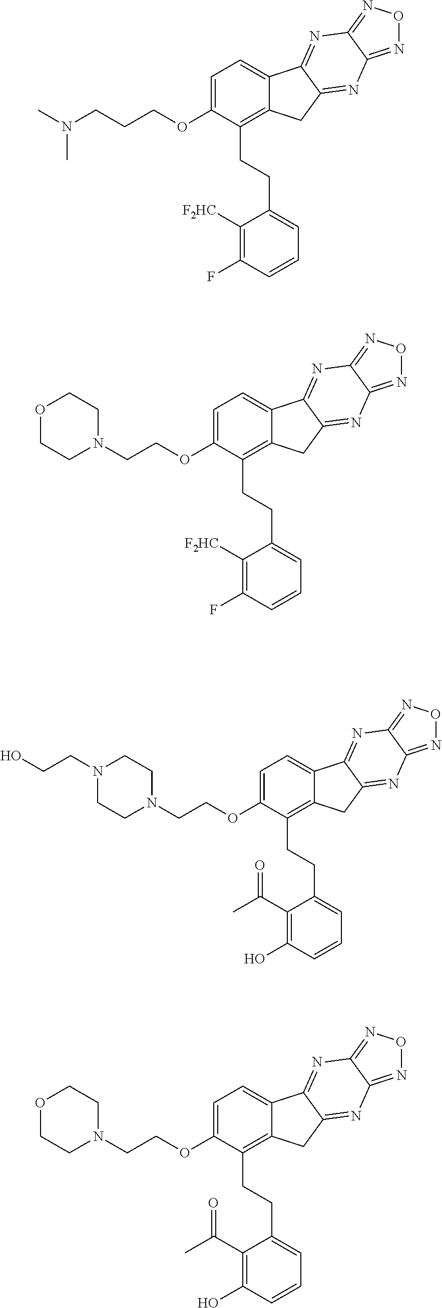 [SMILES: CN(C)CCCOc1ccc2c(c1CCc1cccc(F)c1C(F)F)Cc1nc3nonc3nc1-2 ; Fc1cccc(CCc2c(OCCN3CCOCC3)ccc3c2Cc2nc4nonc4nc2-3)c1C(F)F ; CC(=O)c1c(O)cccc1CCc1c(OCCN2CCN(CCO)CC2)ccc2c1Cc1nc3nonc3nc1-2 ; CC(=O)c1c(O)cccc1CCc1c(OCCN2CCOCC2)ccc2c1Cc1nc3nonc3nc1-2]

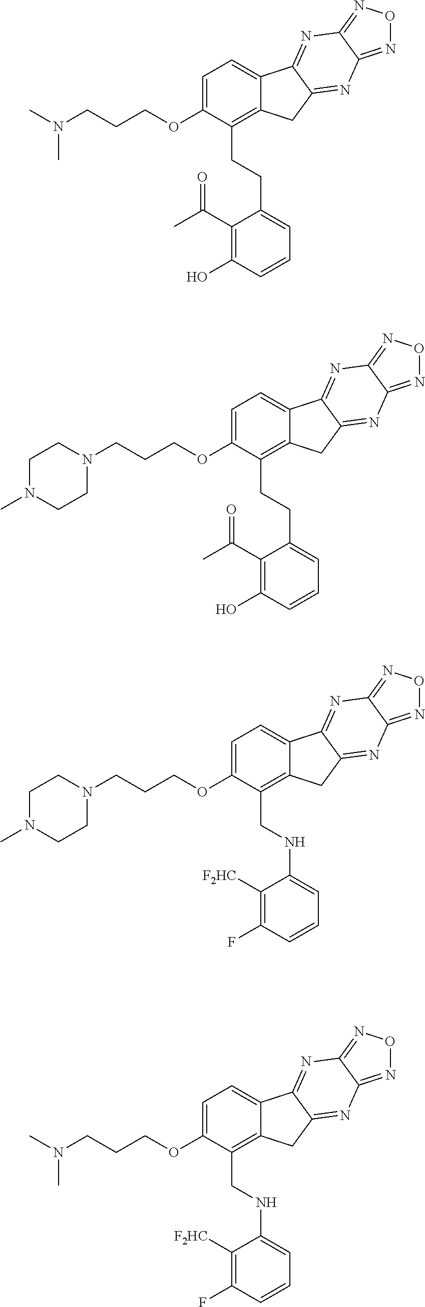 [SMILES: CC(=O)c1c(O)cccc1CCc1c(OCCCN(C)C)ccc2c1Cc1nc3nonc3nc1-2 ; CC(=O)c1c(O)cccc1CCc1c(OCCCN2CCN(C)CC2)ccc2c1Cc1nc3nonc3nc1-2 ; CN1CCN(CCCOc2ccc3c(c2CNc2cccc(F)c2C(F)F)Cc2nc4nonc4nc2-3)CC1 ; CN(C)CCCOc1ccc2c(c1CNc1cccc(F)c1C(F)F)Cc1nc3nonc3nc1-2]

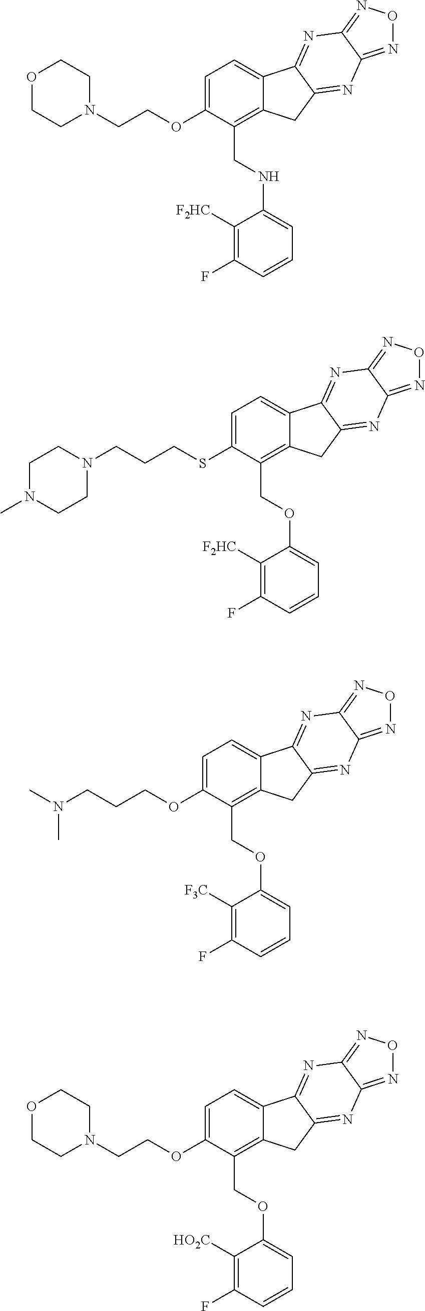 [SMILES: Fc1cccc(NCc2c(OCCN3CCOCC3)ccc3c2Cc2nc4nonc4nc2-3)c1C(F)F ; CN1CCN(CCCSc2ccc3c(c2COc2cccc(F)c2C(F)F)Cc2nc4nonc4nc2-3)CC1 ; CN(C)CCCOc1ccc2c(c1COc1cccc(F)c1C(F)(F)F)Cc1nc3nonc3nc1-2 ; O=C(O)c1c(F)cccc1OCc1c(OCCN2CCOCC2)ccc2c1Cc1nc3nonc3nc1-2]

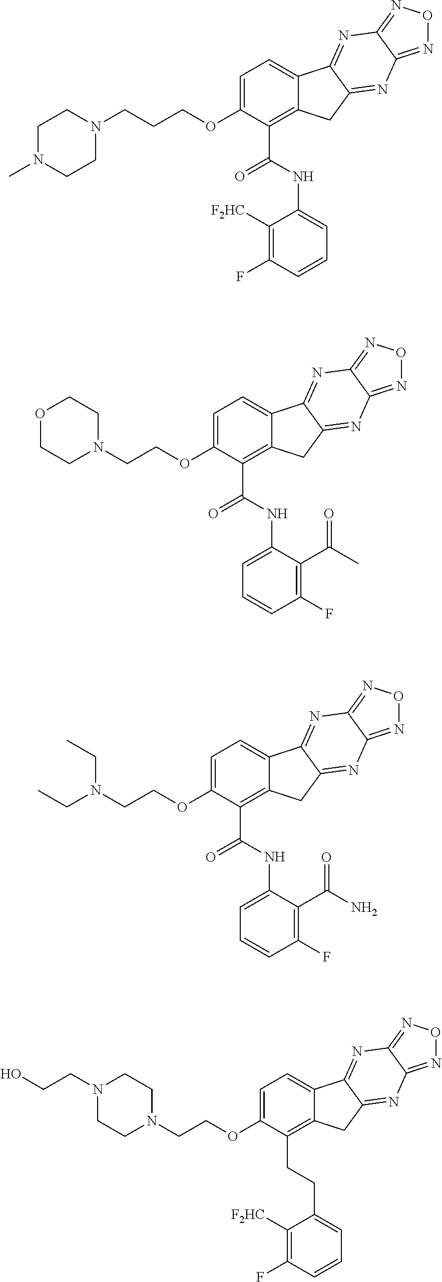 [SMILES: CN1CCN(CCCOc2ccc3c(c2C(=O)Nc2cccc(F)c2C(F)F)Cc2nc4nonc4nc2-3)CC1 ; CC(=O)c1c(F)cccc1NC(=O)c1c(OCCN2CCOCC2)ccc2c1Cc1nc3nonc3nc1-2 ; CCN(CC)CCOc1ccc2c(c1C(=O)Nc1cccc(F)c1C(N)=O)Cc1nc3nonc3nc1-2 ; OCCN1CCN(CCOc2ccc3c(c2CCc2cccc(F)c2C(F)F)Cc2nc4nonc4nc2-3)CC1]

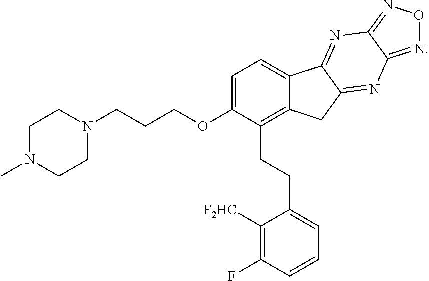 [SMILES: CN1CCN(CCCOc2ccc3c(c2CCc2cccc(F)c2C(F)F)Cc2nc4nonc4nc2-3)CC1]

In some aspects, the cell is a cancer cell. In some aspects, the cell is in a patient. In some aspects, the patient is a cancer patient. In some aspects, the cancer patient has, or does not have, breast cancer, colon cancer, lung cancer, hepatocellular cancer, pancreatic cancer, prostate cancer, glioblastoma, melanoma, or ovarian cancer. In some aspects, the RAL-interacting protein is RLIP76. In some aspects, the RAL-interacting protein inhibitor binds to a transporter substrate binding site of RLIP76. In some aspects, the RAL-interacting protein inhibitor is provided to the cell once, or a multiple of times.

Some aspects of the present disclosure are directed to a compound of formula (I):

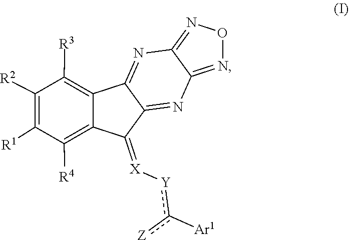

wherein X is N or CH;
Y is NH or O when Z is a carbonyl oxygen, or CH when Z is F;
Z is ═O or F;
R¹ is OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, a substituted or unsubstituted heterocycloalkoxy, or a substituted or unsubstituted heterocyclosulfide;
Ar¹ is

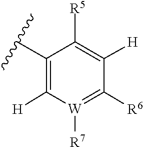

W is N or C;
R² and R⁴ are each independently H, halogen, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkyl-L¹)ₚ, (C₄₋₇ heterocycloalkyl-C₀₋₄ alkylene-L¹)ₚ, (C₆₋₁₀ bridged bicycloheteroalkyl-C₀₋₄ alkylene-L¹)ₚ;
R³ is H, halogen, or C₁₋₆ lower alkyl;
R⁵ is NH₂, OH or F;
R⁶ is H, OH, F, Cl, Br, CH₃, ethynyl, cyano, a substituted or unsubstituted alkenyl, a substituted or unsubstituted alkoxy, or a substituted or unsubstituted sulfide;
R⁷ is nothing when W is N, or H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅-_o bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ when W is C;
R⁹ and R¹⁰ are each independently H, R³, or R⁹ and R¹⁰, and the N atom that connects R⁹ and R¹⁰ can form a heterocyclic ring of 4 to 8 members which can further comprise N, O, S, or SO₂; R¹¹ is C₁₋₆ lower alkyl, C₂₋₆ lower alkenyl, C₂₋₆ lower alkynyl, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
L¹ is independently at each occurrence a bond, O, NH, NR⁴, N(C₁₋₆ lower acyl), S, SO, SO₂, CO, CONR⁴, CO₂, NR⁴CO, OCONH, OCONR⁴, OCO₂, NR⁴CONR⁴, NR⁴CSNR⁴, NR⁴CO, OCO, SO₂NR⁴, or NR⁴SO₂;
p is 1-3, wherein each repeat unit can be the same or different with respect to both cyclic and linear elements; and wherein each of R¹—R¹¹, independently, can optionally be substituted with up to 3 substituents selected from the group consisting of OH, CN, C₁₋₆ lower alkyl, C₁₋₆ lower alkoxy, C₁₋₆ lower alkylthio, C₁₋₆ lower alkylamino, C₁₋₆ lower dialkylamino, C₁₋₆ lower acyloxy, optionally substituted phenyl, optionally substituted C₅₋₆ heteroaryl, halogen, CF₃, OCF₃, oxo, (OCH₂CH₂O)₁₋₁₀OH, and (OCH₂CH₂O)₁₋₁₀OCH₃; or an enantiomer, diastereoisomer, racemic mixture, or salt(s) thereof or salt(s) thereof. It is specifically contemplated that one or more of the substituents listed as being present in certain aspects may be excluded in other aspects. For example, it is specifically contemplated that R¹ is not OH in certain aspects. In some aspects, only one of R¹, R², R³, R⁴ are not hydrogen. In some aspects, R¹, R², R³, R⁴ are all hydrogen. In some aspects, any hydrogen or carbon present in the compound are replaced by a corresponding isotope ²H, ³H, ¹³C or ¹⁴C. In some aspects, the compound of formula (I) is further defined as:

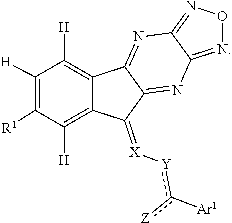

In some aspects, a compound of formula (I) is at least one of the following compounds or is not at least one of the following compounds:

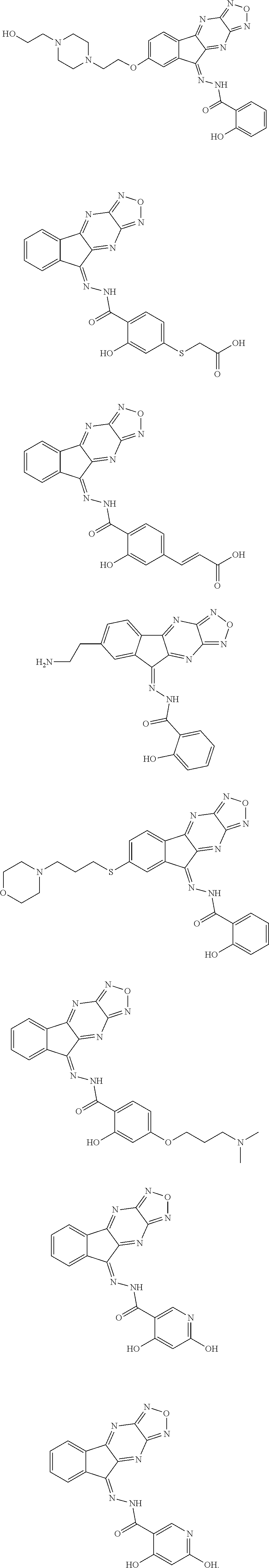

In some aspects, the compound of formula (I) is not:

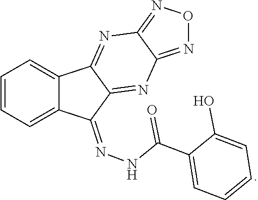 [SMILES: O=C(N/N=C1/c2ccccc2-c2nc3nonc3nc21)c1ccccc1O]

Some aspects of the present disclosure are directed to a compound of formula (II):

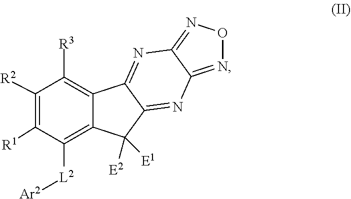

E¹ and E² are independently H or F, or one of E¹ and E² is H and the other OH, CCH or CN, or E¹ and E² may be taken together to be ═O or ═CH₂;
Ar² is

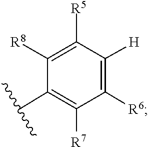

L₂ is —CH₂CH₂—, —CH₂CHR²—, —CH₂NH—, —NHCH₂—, —CH₂NR³—, —NHCHR³—, —CH₂O—, —OCH₂—, —OCHR³—, —COCH₂—, —COCHR²—, —CH₂CO—, —CH₂S—, —CH₂SO—, —CH₂SO(NH)—, —SCH₂—, —SOCH₂—, —SO₂CH₂—, —SO(NH)CH₂—, —SCHR³—, —SOCHR³—, —SO₂CHR³—, —SO(NH)CHR³—, —CH₂SO₂—, —SO₂CHR²—, —SOCHR²—, —SONHCHR²—, —NHS—, —NHSO—, —NHSO₂—, —SNH—, —SONH—, —SO₂NH—, —NHNH—, —SNR³—, —SONR³—, —SO₂NR³—, —NHR³—, —R³NH—, —NHO—, —ONH—, CONH—, —NHCO—, —CONH—, —ONR³—, —NR³CO—, —CONR³—, —OCO— or —COO—;
R¹ is OH, OR⁹, OCOR¹¹, OCOR⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, a substituted or unsubstituted heterocycloalkoxy, or a substituted or unsubstituted heterocyclosulfide;
R² is H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
R³ is C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, with the proviso that the L¹ directly bonded to L² is only selected from a bond, CO, CONR⁴ or CO₂;
R⁴ is H or C₁₋₆ lower alkyl;
R⁵ is NH₂, OH or F;
R⁶ is H, F, Cl, Br, CH₃, ethynyl or cyano;
R⁷ is H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
R⁸ is CH₂OH, CH₂F, CHF₂, CF₃, CHO, CO₂H, CO₂Me, CONH₂, or CONHMe;
R⁹ and R¹⁰ are each independently H, R³, or R⁹ and R¹⁰, and the N atom that connects R⁹ and R¹⁰ can form a heterocyclic ring of 4 to 8 members which can further comprise N, O, S, or SO₂;
R¹¹ is C₁₋₆ lower alkyl, C₂₋₆ lower alkenyl, C₂₋₆ lower alkynyl, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ; L¹ is independently at each occurrence a bond, O, NH, NR⁴, N(C₁₋₆ lower acyl), S, SO, SO₂, CO, CONR⁴, CO₂, NR⁴CO, OCONH, OCONR⁴, OCO₂, NR⁴CONR⁴, NR⁴CSNR⁴, NR⁴CO, OCO, SO₂NR⁴, or NR⁴SO;
p is 1-3, wherein each repeat unit can be the same or different with respect to both cyclic and linear elements; wherein each of R¹—R¹, independently, can optionally be substituted with up to 3 substituents from the following list, OH, CN, C₁₋₆ lower alkyl, C₁₋₆ lower alkoxy, C₁₋₆ lower alkylthio, C₁₋₆ lower alkylamino, C₁₋₆ lower dialkylamino, C₁₋₆ lower acyloxy, optionally substituted phenyl, optionally substituted C₅₋₆ heteroaryl, halogen, CF₃, CF₃O, oxo, (OCH₂CH₂O)₁₋₁₀OH, and (OCH₂CH₂O)₁₋₁₀OCH₃; or an enantiomer, diastereoisomer, racemic mixture, or salt(s) thereof or salt(s) thereof. It is specifically contemplated that one or more of the substituents listed as being present in certain aspects may be excluded in other aspects. For example, it is specifically contemplated that R¹ is not OH in certain aspects. In some aspects, only one of R¹, R², R³, R⁴ are not hydrogen. In some aspects, R¹, R², R³, R⁴ are all hydrogen. In some aspects, any hydrogen or carbon present in the compound are replaced by a corresponding isotope ²H, ³H, ¹³C or ¹⁴C. In some aspects the compound of formula (II) is further defined as:

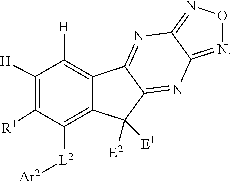

In some aspects, a compound of formula (II) is at least one of the following compounds or is not at least one of the following compounds:

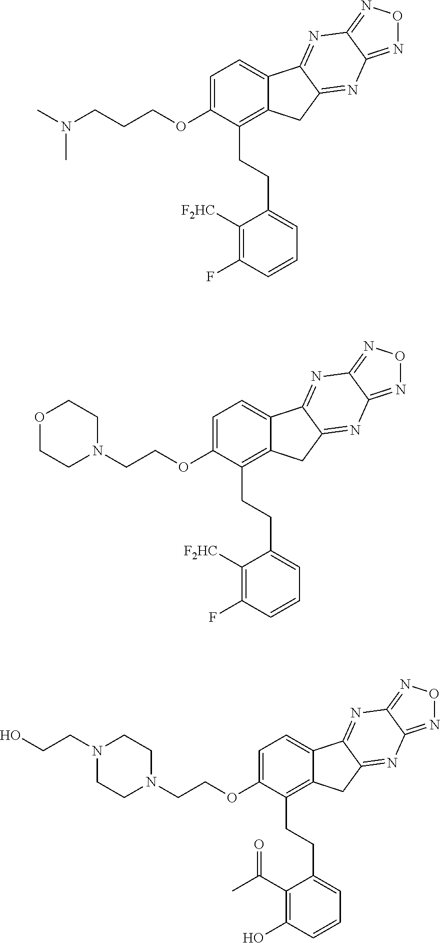 [SMILES: CN(C)CCCOc1ccc2c(c1CCc1cccc(F)c1C(F)F)Cc1nc3nonc3nc1-2 ; Fc1cccc(CCc2c(OCCN3CCOCC3)ccc3c2Cc2nc4nonc4nc2-3)c1C(F)F ; CC(=O)c1c(O)cccc1CCc1c(OCCN2CCN(CCO)CC2)ccc2c1Cc1nc3nonc3nc1-2]

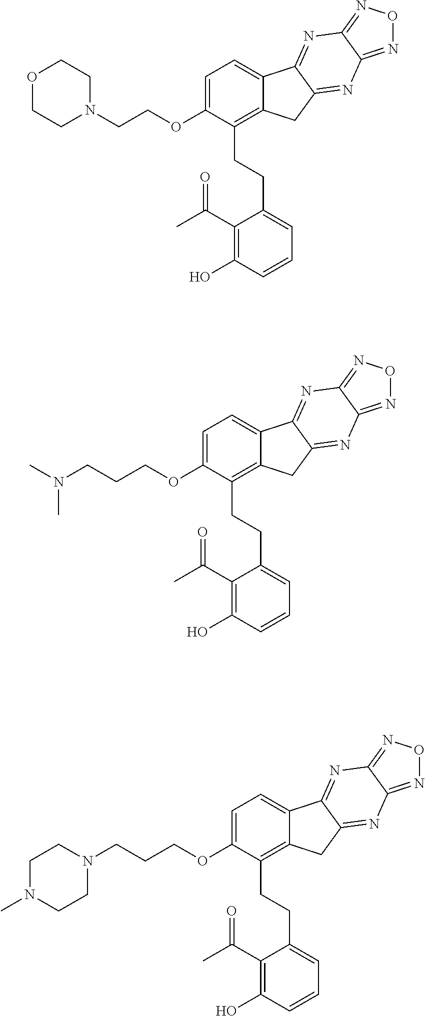 [SMILES: CC(=O)c1c(O)cccc1CCc1c(OCCN2CCOCC2)ccc2c1Cc1nc3nonc3nc1-2 ; CC(=O)c1c(O)cccc1CCc1c(OCCCN(C)C)ccc2c1Cc1nc3nonc3nc1-2 ; CC(=O)c1c(O)cccc1CCc1c(OCCCN2CCN(C)CC2)ccc2c1Cc1nc3nonc3nc1-2]

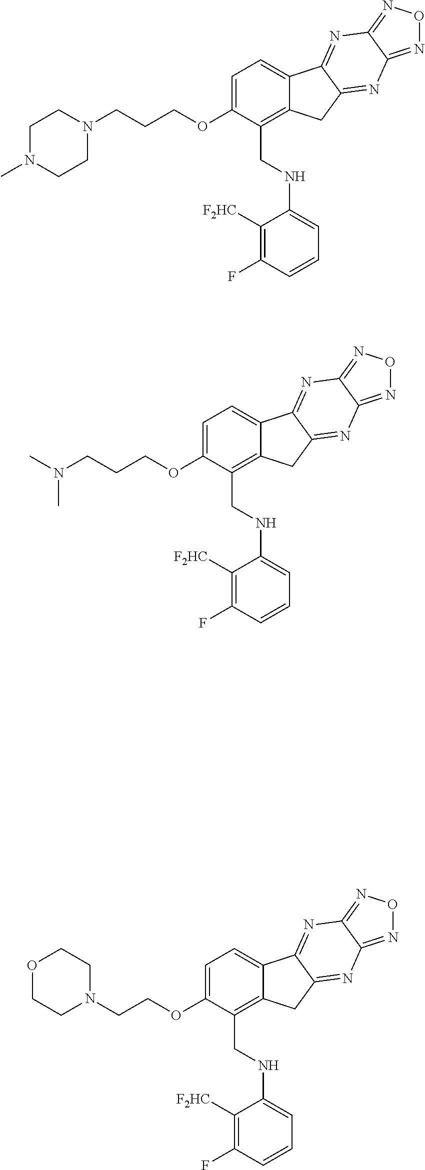 [SMILES: CN1CCN(CCCOc2ccc3c(c2CNc2cccc(F)c2C(F)F)Cc2nc4nonc4nc2-3)CC1 ; CN(C)CCCOc1ccc2c(c1CNc1cccc(F)c1C(F)F)Cc1nc3nonc3nc1-2 ; Fc1cccc(NCc2c(OCCN3CCOCC3)ccc3c2Cc2nc4nonc4nc2-3)c1C(F)F]

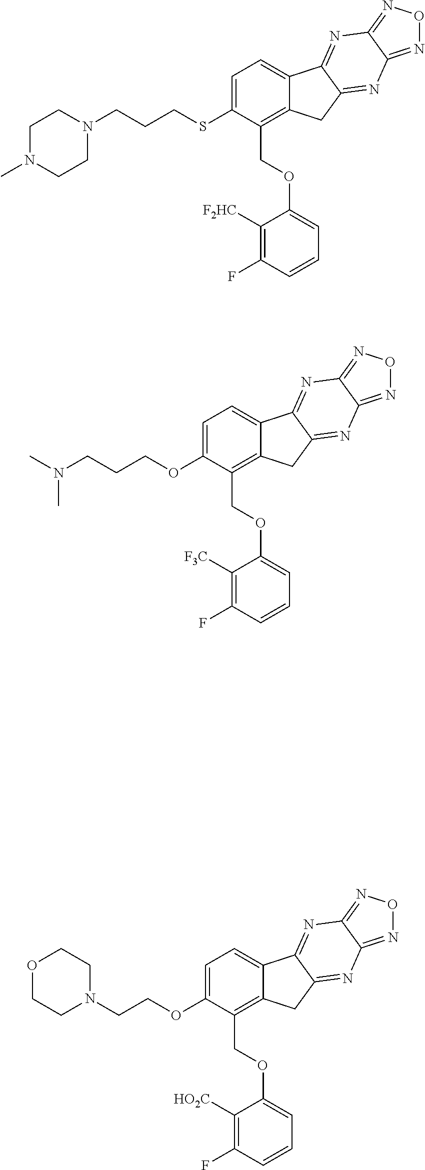 [SMILES: CN1CCN(CCCSc2ccc3c(c2COc2cccc(F)c2C(F)F)Cc2nc4nonc4nc2-3)CC1 ; CN(C)CCCOc1ccc2c(c1COc1cccc(F)c1C(F)(F)F)Cc1nc3nonc3nc1-2 ; O=C(O)c1c(F)cccc1OCc1c(OCCN2CCOCC2)ccc2c1Cc1nc3nonc3nc1-2]

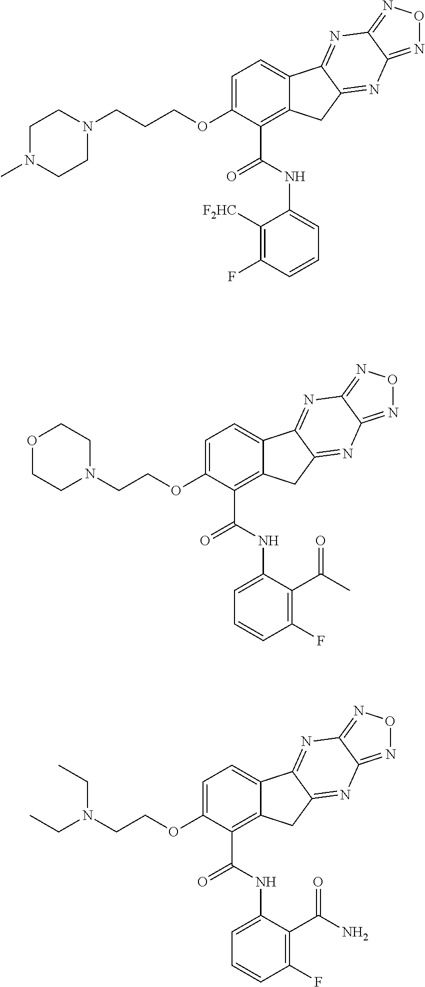 [SMILES: CN1CCN(CCCOc2ccc3c(c2C(=O)Nc2cccc(F)c2C(F)F)Cc2nc4nonc4nc2-3)CC1 ; CC(=O)c1c(F)cccc1NC(=O)c1c(OCCN2CCOCC2)ccc2c1Cc1nc3nonc3nc1-2 ; CCN(CC)CCOc1ccc2c(c1C(=O)Nc1cccc(F)c1C(N)=O)Cc1nc3nonc3nc1-2]

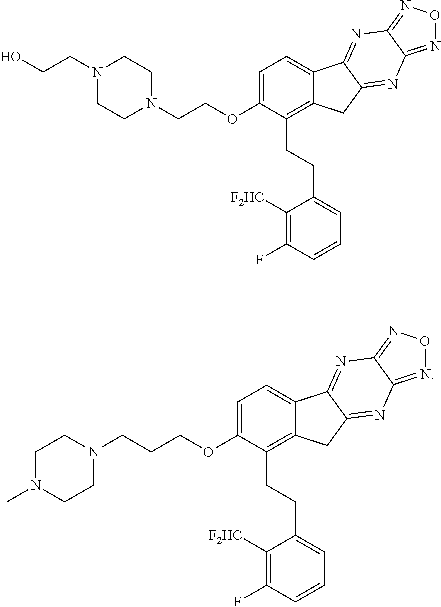 [SMILES: OCCN1CCN(CCOc2ccc3c(c2CCc2cccc(F)c2C(F)F)Cc2nc4nonc4nc2-3)CC1 ; CN1CCN(CCCOc2ccc3c(c2CCc2cccc(F)c2C(F)F)Cc2nc4nonc4nc2-3)CC1]

In some embodiments, the methods and compositions concern a compound that is an inhibitor of a RAL-interacting protein, meaning that the compound directly decreases, inhibits, and/or attenuates the RAL-interacting protein activity when the RAL-interacting protein protein is exposed to the compound. The terms “inhibitor” and “antagonist” are used interchangeably herein. In some aspects, the RAL-interacting protein is RLIP76.

Therefore, embodiments cover a number of methods involving an inhibitor of a RAL-interacting protein, that decrease, inhibit or reduce the RAL-interacting protein activity by or by at least, at most, about, or at 10, 15, 20, 25, 30, 35, 40, 45, 50, 55, 60, 65, 70, 75, 80, 85, 90, 95, or 100% (or any range derivable therein) compared to the RAL-interacting protein activity in the absence of the inhibitor of a RAL-interacting protein. Therefore, in some embodiments, there are methods for inhibiting a RAL-interacting protein in a cell comprising providing to the cell an effective amount of a small molecule that directly inhibits the RAL-interacting protein activity in a cell. In particular embodiments, the RAL-interacting protein inhibitor binds to a transporter substrate binding site of RLIP76 when the inhibitor is incubated with RLIP76.

An “effective amount” of a compound or composition, generally, is defined as that amount sufficient to detectably and repeatedly achieve the stated desired result, for example, to ameliorate, reduce, minimize or limit the extent of the disease or its symptoms or to increase, stimulate, or promote a desirable physiological response. In some embodiments, the stated result may include elimination, eradication or cure of disease.

It is contemplated that in certain embodiments, a cell is a human cell and the subject or patient is a human patient. In other embodiments, a cell is a mammalian cell and the subject or patient is a mammalian patient. It will be understood that different mammals have their own RLIP76 protein that would be a homolog of the corresponding human protein. In certain other embodiments, the cell is a eukaryotic cell, while in other embodiments, the cell is a prokaryotic cell and an RLIP76 protein homolog or analog is the protein that is modulated. In specific embodiments, a cell may be a sex cell, while in others, the cell is a somatic cell. In particular embodiments, cells used in methods of the disclosure may be from a cell line. In certain embodiments, the cell is a cell from or in any organism described herein. Moreover, in some embodiments the cell is a cancer cell, while in other embodiments a cell is non-cancerous or normal. Furthermore, it is contemplated that a cell can be in a patient. Additionally, a cell may be an embryonic stem (ES) cell, such as a murine ES cell, which are used for generating knockout mice. Alternatively, cells may be murine cells that are used for generating a transgenic mouse. Other transgenic animals can be generated using a particular animals cells in the context of methods of the disclosure.

A “disease” is defined as a pathological condition of a body part, an organ, or a system resulting from any cause, such as infection, genetic defect, or environmental stress. A “health-related condition” is defined herein to refer to a condition of a body part, an organ, or a system that may not be pathological, but for which treatment is sought. Examples include conditions for which cosmetic therapy is sought, such as skin wrinkling, skin blemishes, and the like. The disease can be any disease, and non-limiting examples include hyperproliferative diseases such as cancer and premalignant lesions, wounds, and infections.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition.

The terms “treating”, “inhibiting”, or “reducing” or or any variation of these terms, when used in the claims and/or the specification include any measurable decrease or complete inhibition to achieve a desired result.

“Subject, “individual,” and “patient” are used interchangeably and can refer to a human or non-human. In some aspects, the subject can be a human or animal that has cancer but has not been diagnosed with having cancer. In some aspects, the subject can be a human or animal that has cancer and has been diagnosed as having cancer. In some aspects, the subject can be a human or animal that does not have cancer but has previously had or previously been diagnosed as having cancer (e.g., a subject in which cancer is in remission).

It is specifically contemplated that any limitation discussed with respect to one embodiment of the disclosure may apply to any other embodiment of the disclosure. Furthermore, any composition of the disclosure may be used in any method of the disclosure, and any method of the disclosure may be used to produce or to utilize any composition of the disclosure.

The use of the term “or” in the claims is used to mean “and/or” unless explicitly indicated to refer to alternatives only or the alternative are mutually exclusive, although the disclosure supports a definition that refers to only alternatives and “and/or.”

The terms “about” or “approximately” are defined as being close to as understood by one of ordinary skill in the art. In one non-limiting embodiment, the terms are defined to be within 10%, alternatively within 5%, alternatively within 1%, and alternatively within 0.5%.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

The terms “wt. %,” “vol. %,” or “mol. %” refer to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component. The terms “ppm” refer to parts per million by weight of a component, based on the total weight, that includes the component.

The term “substantially” and its variations are defined to include ranges within 10%, within 5%, within 1%, or within 0.5%.

The phrase “and/or” can include “and” or “or.” To illustrate, X, Y, and/or Z can include: X alone, Y alone, Z alone, a combination of X and Y, a combination of X and Z, a combination of Y and Z, or a combination of X, Y, and Z.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compounds, compositions, and methods of the present disclosure can “comprise,” “consist essentially of,” or “consist of” particular ingredients, compounds, components, compositions, steps, etc., disclosed throughout the specification. With respect to the transitional phrase “consisting essentially of,” in one non-limiting aspect, a basic and novel characteristic of the compounds, compositions, and methods of the present disclosure include the treatment or prevention of cancer in a subject, including, but not limited to, reducing RAL-interacting protein (e.g., RLIP76) in a cell.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1. A reaction scheme for synthesizing a compound of formula (I).

FIG. 2. A series of compounds that can be used to synthesize various compounds of formula (I) and formula (II).

FIG. 3. A reaction scheme for synthesizing a compound of formula (I).

FIG. 4. A reaction scheme for synthesizing a compound of formula (I).

FIG. 5. A reaction scheme for synthesizing a compound of formula (I).

FIG. 6. A reaction scheme for synthesizing a compound of formula (II).

FIG. 7. A series of compounds of formula (II) that can be synthesized using a reaction scheme as disclosed herein.

FIG. 8. A reaction scheme for synthesizing a compound of formula (II).

FIG. 9. A series of compounds of formula (II) that can be synthesized using a reaction scheme as disclosed herein.

FIG. 10. A reaction scheme for synthesizing a compound of formula (II).

FIG. 11. A series of compounds of formula (II) that can be synthesized using a reaction scheme as disclosed herein.

FIG. 12. A reaction scheme for synthesizing a compound of formula (II).

FIG. 13. A series of compounds of formula (II) that can be synthesized using a reaction scheme as disclosed herein.

FIG. 14. A reaction scheme for synthesizing a compound of formula (II).

FIG. 15. A series of compounds of formula (II) that can be synthesized using a reaction scheme as disclosed herein.

FIG. 16. A panel of cancer cell lines treated with AVE-001 at various concentrations.

FIG. 17. A panel of cancer cell lines treated with AVE-005 at various concentrations.

FIG. 18. A panel of cancer cell lines treated with AVE-006 at various concentrations.

FIG. 19. A panel of cancer cell lines treated with AVE-007 at various concentrations.

### DESCRIPTION OF ILLUSTRATIVE EMBODIMENTS

RLIP76, also known as RalBP1, is a multifunctional membrane protein that transports glutathione conjugates of electrophilic compounds and other xenobiotics including chemotherapy agents out of cells. The protein is overexpressed in lung carcinomas, ovarian carcinomas, and melanomas. The protein also binds Ral and participates in mitotic spindle function, clathrin-dependent endocytosis, and triggers GTPase-activating protein activity. It is found throughout the cell, in membrane, cytosol, and the nucleus, and is known to shift between these compartments in response to stress. Loss of RLIP76 by antibody or antisense therapy is associated with increased sensitivity to radiation and chemotherapy.

Taking into consideration the selective over-expression of RLIP76 in cancer cells which translates to proportional amount of drug-efflux, the signaling function of RLIP76 and the dependence of cancer cells on this protein to overcome chemo/radio-therapy induced oxidant injury makes it one of the key proteins involved in cancer cell survival, proliferation, and/or metastasis. This emphasizes the importance of targeting RLIP76 expression or activity to help treat or prevent cancer in subjects.

### A. CANCER

Cancer cells that may be treated by methods and compositions of the disclosure also include, or exclude, cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### B. CHEMICAL DEFINITIONS

As used herein, a “small molecule” refers to an organic compound that is either synthesized via conventional organic chemistry methods (e.g., in a laboratory) or found in nature. Typically, a small molecule is characterized in that it contains several carbon-carbon bonds, and has a molecular weight of less than about 1500 grams/mole. In certain embodiments, small molecules are less than about 1000 grams/mole. In certain embodiments, small molecules are less than about 550 grams/mole. In certain embodiments, small molecules are between about 200 and about 550 grams/mole. In certain embodiments, small molecules exclude peptides (e.g., compounds comprising 2 or more amino acids joined by a peptidyl bond). In certain embodiments, small molecules exclude nucleic acids.

As used herein, the term “amino” means —NH₂; the term “nitro” means —NO₂; the term “halo” or “halogen” designates —F, —Cl, —Br or —I; the term “mercapto” means —SH; the term “cyano” means —CN; the term “azido” means —N₃; the term “silyl” means —SiH₃, and the term “hydroxy” means —OH. In certain embodiments, a halogen may be —Br or —I.

As used herein, a “monovalent anion” refers to anions of a −1 charge. Such anions are well-known to those of skill in the art. Non-limiting examples of monovalent anions include halides (e.g., F⁻, Cl⁻, Br⁻ and I⁻), NO₂⁻, NO₃⁻, hydroxide (OH⁻) and azide (N₃⁻).

As used herein, the structure [symbol] indicates that the bond may be a single bond or a double bond. Those of skill in the chemical arts understand that in certain circumstances, a double bond between two particular atoms is chemically feasible and in certain circumstances, a double bond is not. The present disclosure therefore contemplates that a double bond may be formed only when chemically feasible.

The term “alkyl” includes straight-chain alkyl, branched-chain alkyl, cycloalkyl (alicyclic), cyclic alkyl, heteroatom-unsubstituted alkyl, heteroatom-substituted alkyl, heteroatom-unsubstituted Cₙ-alkyl, and heteroatom-substituted Cₙ-alkyl. In certain embodiments, lower alkyls are contemplated. The term “lower alkyl” refers to alkyls of 1-6 carbon atoms (that is, 1, 2, 3, 4, 5 or 6 carbon atoms). The term “heteroatom-unsubstituted Cₙ-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, and cyclohexyl, are all non-limiting examples of heteroatom-unsubstituted alkyl groups. The term “heteroatom-substituted Cₙ-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all non-limiting examples of heteroatom-substituted alkyl groups: trifluoromethyl, —CH₂F, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The term “alkenyl” includes straight-chain alkenyl, branched-chain alkenyl, cycloalkenyl, cyclic alkenyl, heteroatom-unsubstituted alkenyl, heteroatom-substituted alkenyl, heteroatom-unsubstituted Cₙ-alkenyl, and heteroatom-substituted Cₙ-alkenyl. In certain embodiments, lower alkenyls are contemplated. The term “lower alkenyl” refers to alkenyls of 1-6 carbon atoms (that is, 1, 2, 3, 4, 5 or 6 carbon atoms). The term “heteroatom-unsubstituted Cₙ-alkenyl” refers to a radical, having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C₂-C₁₀-alkenyl has 2 to 10 carbon atoms. Heteroatom-unsubstituted alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “heteroatom-substituted Cₙ-alkenyl” refers to a radical, having a single nonaromatic carbon atom as the point of attachment and at least one nonaromatic carbon-carbon double bond, but no carbon-carbon triple bonds, further having a linear or branched, cyclic or acyclic structure, further having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkenyl has 2 to 10 carbon atoms. The groups, —CH═CHF, —CH═CHCl and —CH═CHBr, are non-limiting examples of heteroatom-substituted alkenyl groups.

The term “aryl” includes heteroatom-unsubstituted aryl, heteroatom-substituted aryl, heteroatom-unsubstituted Cₙ-aryl, heteroatom-substituted Cₙ-aryl, heteroaryl, heterocyclic aryl groups, carbocyclic aryl groups, biaryl groups, and single-valent radicals derived from polycyclic fused hydrocarbons (PAHs). The term “heteroatom-unsubstituted Cₙ-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C₆-C₁₀-aryl has 6 to 10 carbon atoms. Non-limiting examples of heteroatom-unsubstituted aryl groups include phenyl (Ph), methylphenyl, (dimethyl)phenyl, —C₆H₄CH₂CH₃, —C₆H₄CH₂CH₂CH₃, —C₆H₄CH(CH₃)₂, —C₆H₄CH(CH₂)₂, —C₆H₃(CH₃)CH₂CH₃, —C₆H₄CH═CH₂, —C₆H₄CH═CHCH₃, —C₆H₄C≡CH, —C₆H₄C≡CCH₃, naphthyl, and the radical derived from biphenyl. The term “heteroatom-substituted Cₙ-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. Non-limiting examples of heteroatom-substituted aryl groups include the groups: —C₆H₄F, —C₆H₄Cl, —C₆H₄Br, —C₆H₄I, —C₆H₄₀H, —C₆H₄OCH₃, —C₆H₄OCH₂CH₃, —C₆H₄₀C(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, —C₆H₄CON(CH₃)₂, furanyl, thienyl, pyridyl, pyrrolyl, pyrimidyl, pyrazinyl, quinolyl, indolyl, and imidazoyl. In certain embodiments, heteroatom-substituted aryl groups are contemplated. In certain embodiments, heteroatom-unsubstituted aryl groups are contemplate. In certain embodiments, an aryl group may be mono-, di-, tri-, tetra- or penta-substituted with one or more heteroatom-containing substitutents.

The term “aralkyl” includes heteroatom-unsubstituted aralkyl, heteroatom-substituted aralkyl, heteroatom-unsubstituted Cₙ-aralkyl, heteroatom-substituted Cₙ-aralkyl, heteroaralkyl, and heterocyclic aralkyl groups. In certain embodiments, lower aralkyls are contemplated. The term “lower aralkyl” refers to aralkyls of 7-12 carbon atoms (that is, 7, 8, 9, 10, 11 or 12 carbon atoms). The term “heteroatom-unsubstituted Cₙ-aralkyl” refers to a radical, having a single saturated carbon atom as the point of attachment, further having a total of n carbon atoms, wherein at least 6 of the carbon atoms form an aromatic ring structure containing only carbon atoms, 7 or more hydrogen atoms, and no heteroatoms. For example, a heteroatom-unsubstituted C₇-C₁₀-aralkyl has 7 to 10 carbon atoms. Non-limiting examples of heteroatom-unsubstituted aralkyls are: phenylmethyl (benzyl, Bn) and phenylethyl. The term “heteroatom-substituted Cₙ-aralkyl” refers to a radical, having a single saturated carbon atom as the point of attachment, further having a total of n carbon atoms, 0, 1, or more than one hydrogen atom, and at least one heteroatom, wherein at least one of the carbon atoms is incorporated an aromatic ring structures, further wherein each heteroatom is independently selected from the group consisting of N, O, F, Cl, Br, I, Si, P, and S. For example, a heteroatom-substituted C₂-C₁₀-heteroaralkyl has 2 to 10 carbon atoms.

The term “acyl” includes straight-chain acyl, branched-chain acyl, cycloacyl, cyclic acyl, heteroatom-unsubstituted acyl, heteroatom-substituted acyl, heteroatom-unsubstituted Cₙ-acyl, heteroatom-substituted Cₙ-acyl, alkylcarbonyl, alkoxycarbonyl and aminocarbonyl groups. In certain embodiments, lower acyls are contemplated. The term “lower acyl” refers to acyls of 1-6 carbon atoms (that is, 1, 2, 3, 4, 5 or 6 carbon atoms). The term “heteroatom-unsubstituted Cₙ-acyl” refers to a radical, having a single carbon atom of a carbonyl group as the point of attachment, further having a linear or branched, cyclic or acyclic structure, further having a total of n carbon atoms, 1 or more hydrogen atoms, a total of one oxygen atom, and no additional heteroatoms. For example, a heteroatom-unsubstituted C₁-C₁₀-acyl has 1 to 10 carbon atoms. The groups, —CHO, —C(O)CH₃, —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, and —COC₆H₃(CH₃)₂, are non-limiting examples of heteroatom-unsubstituted acyl groups. The term “heteroatom-substituted Cₙ-acyl” refers to a radical, having a single carbon atom as the point of attachment, the carbon atom being part of a carbonyl group, further having a linear or branched, cyclic or acyclic structure, further having a total of n carbon atoms, 0, 1, or more than one hydrogen atom, at least one additional heteroatom, in addition to the oxygen of the carbonyl group, wherein each additional heteroatom is independently selected from the group consisting of N, O, F, Cl, Br, I, Si, P, and S. For example, a heteroatom-substituted C₁-C₁₀-acyl has 1 to 10 carbon atoms. The groups, —C(O)CH₂CF₃, —CO₂H, —CO₂—, —CO₂CH₃, —CO₂CH₂CH₃, —CO₂CH₂CH₂CH₃, —CO₂CH(CH₃)₂, —CO₂CH(CH₂)₂, —C(O)NH₂ (carbamoyl), —C(O)NHCH₃, —C(O)NHCH₂CH₃, —CONHCH(CH₃)₂, —CONHCH(CH₂)₂, —CON(CH₃)₂, and —CONHCH₂CF₃, are non-limiting examples of heteroatom-substituted acyl groups.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted Cₙ-alkoxy, and heteroatom-substituted Cₙ-alkoxy. In certain embodiments, lower alkoxys are contemplated. The term “lower alkoxy” refers to alkoxys of 1-6 carbon atoms (that is, 1, 2, 3, 4, 5 or 6 carbon atoms). The term “heteroatom-unsubstituted Cₙ-alkoxy” refers to a group, having the structure —OR, in which R is a heteroatom-unsubstituted Cₙ-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂. The term “heteroatom-substituted Cₙ-alkoxy” refers to a group, having the structure —OR, in which R is a heteroatom-substituted Cₙ-alkyl, as that term is defined above. For example, —OCH₂CF₃ is a heteroatom-substituted alkoxy group.

The term “alkenyloxy” includes straight-chain alkenyloxy, branched-chain alkenyloxy, cycloalkenyloxy, cyclic alkenyloxy, heteroatom-unsubstituted alkenyloxy, heteroatom-substituted alkenyloxy, heteroatom-unsubstituted Cₙ-alkenyloxy, and heteroatom-substituted Cₙ-alkenyloxy. The term “heteroatom-unsubstituted Cₙ-alkenyloxy” refers to a group, having the structure —OR, in which R is a heteroatom-unsubstituted Cₙ-alkenyl, as that term is defined above. The term “heteroatom-substituted Cₙ-alkenyloxy” refers to a group, having the structure —OR, in which R is a heteroatom-substituted Cₙ-alkenyl, as that term is defined above.

The term “alkynyloxy” includes straight-chain alkynyloxy, branched-chain alkynyloxy, cycloalkynyloxy, cyclic alkynyloxy, heteroatom-unsubstituted alkynyloxy, heteroatom-substituted alkynyloxy, heteroatom-unsubstituted Cₙ-alkynyloxy, and heteroatom-substituted Cₙ-alkynyloxy. The term “heteroatom-unsubstituted Cₙ-alkynyloxy” refers to a group, having the structure —OR, in which R is a heteroatom-unsubstituted Cₙ-alkynyl, as that term is defined above. The term “heteroatom-substituted Cₙ-alkynyloxy” refers to a group, having the structure —OR, in which R is a heteroatom-substituted Cₙ-alkynyl, as that term is defined above.

The term “aryloxy” includes heteroatom-unsubstituted aryloxy, heteroatom-substituted aryloxy, heteroatom-unsubstituted Cₙ-aryloxy, heteroatom-substituted Cₙ-aryloxy, heteroaryloxy, and heterocyclic aryloxy groups. The term “heteroatom-unsubstituted Cₙ-aryloxy” refers to a group, having the structure —OAr, in which Ar is a heteroatom-unsubstituted Cₙ-aryl, as that term is defined above. A non-limiting example of a heteroatom-unsubstituted aryloxy group is —OC₆H₅. The term “heteroatom-substituted Cₙ-aryloxy” refers to a group, having the structure —OAr, in which Ar is a heteroatom-substituted Cₙ-aryl, as that term is defined above.

The term “aralkyloxy” includes heteroatom-unsubstituted aralkyloxy, heteroatom-substituted aralkyloxy, heteroatom-unsubstituted Cₙ-aralkyloxy, heteroatom-substituted Cₙ-aralkyloxy, heteroaralkyloxy, and heterocyclic aralkyloxy groups. The term “heteroatom-unsubstituted Cₙ-aralkyloxy” refers to a group, having the structure —OAr, in which Ar is a heteroatom-unsubstituted Cₙ-aralkyl, as that term is defined above. The term “heteroatom-substituted Cₙ-aralkyloxy” refers to a group, having the structure —OAr, in which Ar is a heteroatom-substituted Cₙ-aralkyl, as that term is defined above.

The term “acyloxy” includes straight-chain acyloxy, branched-chain acyloxy, cycloacyloxy, cyclic acyloxy, heteroatom-unsubstituted acyloxy, heteroatom-substituted acyloxy, heteroatom-unsubstituted Cₙ-acyloxy, heteroatom-substituted Cₐ-acyloxy, alkylcarbonyloxy, arylcarbonyloxy, alkoxycarbonyloxy, aryloxycarbonyloxy, and carboxylate groups. The term “heteroatom-unsubstituted Cₙ-acyloxy” refers to a group, having the structure —OAc, in which Ac is a heteroatom-unsubstituted Cₙ-acyl, as that term is defined above. For example, —OC(O)CH₃ is a non-limiting example of a heteroatom-unsubstituted acyloxy group. The term “heteroatom-substituted C-acyloxy” refers to a group, having the structure —OAc, in which Ac is a heteroatom-substituted Cₙ-acyl, as that term is defined above. For example, —OC(O)OCH₃ and —OC(O)NHCH₃ are non-limiting examples of heteroatom-unsubstituted acyloxy groups.

The term “alkylamino” includes straight-chain alkylamino, branched-chain alkylamino, cycloalkylamino, cyclic alkylamino, heteroatom-unsubstituted alkylamino, heteroatom-substituted alkylamino, heteroatom-unsubstituted Cₙ-alkylamino, and heteroatom-substituted Cₙ-alkylamino. The term “heteroatom-unsubstituted Cₙ-alkylamino” refers to a radical, having a single nitrogen atom as the point of attachment, further having one or two saturated carbon atoms attached to the nitrogen atom, further having a linear or branched, cyclic or acyclic structure, containing a total of n carbon atoms, all of which are nonaromatic, 4 or more hydrogen atoms, a total of 1 nitrogen atom, and no additional heteroatoms. For example, a heteroatom-unsubstituted C₁-C₁₀-alkylamino has 1 to 10 carbon atoms. The term “heteroatom-unsubstituted Cₙ-alkylamino” includes groups, having the structure —NHR, in which R is a heteroatom-unsubstituted Cₙ-alkyl, as that term is defined above. A heteroatom-unsubstituted alkylamino group would include —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —N(CH₃)₂, —N(CH₃)CH₂CH₃, —N(CH₂CH₃)₂, N-pyrrolidinyl, and N-piperidinyl. The term “heteroatom-substituted Cₙ-alkylamino” refers to a radical, having a single nitrogen atom as the point of attachment, further having one or two saturated carbon atoms attached to the nitrogen atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₁-C₁₀-alkylamino has 1 to 10 carbon atoms. The term “heteroatom-substituted Cₙ-alkylamino” includes groups, having the structure —NHR, in which R is a heteroatom-substituted Cₙ-alkyl, as that term is defined above.

The term “alkenylamino” includes straight-chain alkenylamino, branched-chain alkenylamino, cycloalkenylamino, cyclic alkenylamino, heteroatom-unsubstituted alkenylamino, heteroatom-substituted alkenylamino, heteroatom-unsubstituted Cₙ-alkenylamino, heteroatom-substituted Cₙ-alkenylamino, dialkenylamino, and alkyl(alkenyl)amino groups. The term “heteroatom-unsubstituted Cₙ-alkenylamino” refers to a radical, having a single nitrogen atom as the point of attachment, further having one or two carbon atoms attached to the nitrogen atom, further having a linear or branched, cyclic or acyclic structure, containing at least one nonaromatic carbon-carbon double bond, a total of n carbon atoms, 4 or more hydrogen atoms, a total of one nitrogen atom, and no additional heteroatoms. For example, a heteroatom-unsubstituted C₂-C₁₀-alkenylamino has 2 to 10 carbon atoms. The term “heteroatom-unsubstituted Cₙ-alkenylamino” includes groups, having the structure —NHR, in which R is a heteroatom-unsubstituted Cₙ-alkenyl, as that term is defined above. The term “heteroatom-substituted Cₙ-alkenylamino” refers to a radical, having a single nitrogen atom as the point of attachment and at least one nonaromatic carbon-carbon double bond, but no carbon-carbon triple bonds, further having one or two carbon atoms attached to the nitrogen atom, further having a linear or branched, cyclic or acyclic structure, further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkenylamino has 2 to 10 carbon atoms. The term “heteroatom-substituted Cₙ-alkenylamino” includes groups, having the structure —NHR, in which R is a heteroatom-substituted Cₙ-alkenyl, as that term is defined above.

The term “alkynylamino” includes straight-chain alkynylamino, branched-chain alkynylamino, cycloalkynylamino, cyclic alkynylamino, heteroatom-unsubstituted alkynylamino, heteroatom-substituted alkynylamino, heteroatom-unsubstituted Cₙ-alkynylamino, heteroatom-substituted Cₙ-alkynylamino, dialkynylamino, alkyl(alkynyl)amino, and alkenyl(alkynyl)amino groups. The term “heteroatom-unsubstituted Cₙ-alkynylamino” refers to a radical, having a single nitrogen atom as the point of attachment, further having one or two carbon atoms attached to the nitrogen atom, further having a linear or branched, cyclic or acyclic structure, containing at least one carbon-carbon triple bond, a total of n carbon atoms, at least one hydrogen atoms, a total of one nitrogen atom, and no additional heteroatoms. For example, a heteroatom-unsubstituted C₂-C₁₀-alkynylamino has 2 to 10 carbon atoms. The term “heteroatom-unsubstituted Cₙ-alkynylamino” includes groups, having the structure —NHR, in which R is a heteroatom-unsubstituted Cₙ-alkynyl, as that term is defined above. The term “heteroatom-substituted Cₙ-alkynylamino” refers to a radical, having a single nitrogen atom as the point of attachment, further having one or two carbon atoms attached to the nitrogen atom, further having at least one nonaromatic carbon-carbon triple bond, further having a linear or branched, cyclic or acyclic structure, and further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkynylamino has 2 to 10 carbon atoms. The term “heteroatom-substituted Cₙ-alkynylamino” includes groups, having the structure —NHR, in which R is a heteroatom-substituted Cₙ-alkynyl, as that term is defined above.

The term “arylamino” includes heteroatom-unsubstituted arylamino, heteroatom-substituted arylamino, heteroatom-unsubstituted Cₙ-arylamino, heteroatom-substituted Cₙ-arylamino, heteroarylamino, heterocyclic arylamino, and alkyl(aryl)amino groups. The term “heteroatom-unsubstituted Cₙ-arylamino” refers to a radical, having a single nitrogen atom as the point of attachment, further having at least one aromatic ring structure attached to the nitrogen atom, wherein the aromatic ring structure contains only carbon atoms, further having a total of n carbon atoms, 6 or more hydrogen atoms, a total of one nitrogen atom, and no additional heteroatoms. For example, a heteroatom-unsubstituted C₆-C₁₀-arylamino has 6 to 10 carbon atoms. The term “heteroatom-unsubstituted Cₙ-arylamino” includes groups, having the structure —NHR, in which R is a heteroatom-unsubstituted Cₙ-aryl, as that term is defined above. The term “heteroatom-substituted Cₙ-arylamino” refers to a radical, having a single nitrogen atom as the point of attachment, further having a total of n carbon atoms, at least one hydrogen atom, at least one additional heteroatoms, that is, in addition to the nitrogen atom at the point of attachment, wherein at least one of the carbon atoms is incorporated into one or more aromatic ring structures, further wherein each additional heteroatom is independently selected from the group consisting of N, O, F, Cl, Br, I, Si, P, and S. For example, a heteroatom-substituted C₆-C₁₀-arylamino has 6 to 10 carbon atoms. The term “heteroatom-substituted Cₙ-arylamino” includes groups, having the structure —NHR, in which R is a heteroatom-substituted Cₙ-aryl, as that term is defined above.

The term “aralkylamino” includes heteroatom-unsubstituted aralkylamino, heteroatom-substituted aralkylamino, heteroatom-unsubstituted Cₐ-aralkylamino, heteroatom-substituted Cₙ-aralkylamino, heteroaralkylamino, heterocyclic aralkylamino groups, and diaralkylamino groups. The term “heteroatom-unsubstituted Cₙ-aralkylamino” refers to a radical, having a single nitrogen atom as the point of attachment, further having one or two saturated carbon atoms attached to the nitrogen atom, further having a total of n carbon atoms, wherein at least 6 of the carbon atoms form an aromatic ring structure containing only carbon atoms, 8 or more hydrogen atoms, a total of one nitrogen atom, and no additional heteroatoms. For example, a heteroatom-unsubstituted C₇-C₁₀-aralkylamino has 7 to 10 carbon atoms. The term “heteroatom-unsubstituted Cₙ-aralkylamino” includes groups, having the structure —NHR, in which R is a heteroatom-unsubstituted Cₙ-aralkyl, as that term is defined above. The term “heteroatom-substituted Cₙ-aralkylamino” refers to a radical, having a single nitrogen atom as the point of attachment, further having at least one or two saturated carbon atoms attached to the nitrogen atom, further having a total of n carbon atoms, 0, 1, or more than one hydrogen atom, at least one additional heteroatom, that is, in addition to the nitrogen atom at the point of attachment, wherein at least one of the carbon atom incorporated into an aromatic ring, further wherein each heteroatom is independently selected from the group consisting of N, O, F, Cl, Br, I, Si, P, and S. For example, a heteroatom-substituted C₇-C₁₀-aralkylamino has 7 to 10 carbon atoms. The term “heteroatom-substituted Cₙ-aralkylamino” includes groups, having the structure —NHR, in which R is a heteroatom-substituted Cₙ-aralkyl, as that term is defined above.

The term “amido” includes straight-chain amido, branched-chain amido, cycloamido, cyclic amido, heteroatom-unsubstituted amido, heteroatom-substituted amido, heteroatom-unsubstituted Cₙ-amido, heteroatom-substituted Cₙ-amido, alkylcarbonylamino, arylcarbonylamino, alkoxycarbonylamino, aryloxycarbonylamino, acylamino, alkylaminocarbonylamino, arylaminocarbonylamino, and ureido groups. The term “heteroatom-unsubstituted Cₐ-amido” refers to a radical, having a single nitrogen atom as the point of attachment, further having a carbonyl group attached via its carbon atom to the nitrogen atom, further having a linear or branched, cyclic or acyclic structure, further having a total of n carbon atoms, 1 or more hydrogen atoms, a total of one oxygen atom, a total of one nitrogen atom, and no additional heteroatoms. For example, a heteroatom-unsubstituted C₁-C₁₀-amido has 1 to 10 carbon atoms. The term “heteroatom-unsubstituted Cₙ-amido” includes groups, having the structure —NHR, in which R is a heteroatom-unsubstituted Cₙ-acyl, as that term is defined above. The group, —NHC(O)CH₃, is a non-limiting example of a heteroatom-unsubstituted amido group. The term “heteroatom-substituted Cₙ-amido” refers to a radical, having a single nitrogen atom as the point of attachment, further having a carbonyl group attached via its carbon atom to the nitrogen atom, further having a linear or branched, cyclic or acyclic structure, further having a total of n aromatic or nonaromatic carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C₁₀-amido has 1 to 10 carbon atoms. The term “heteroatom-substituted Cₙ-amido” includes groups, having the structure —NHR, in which R is a heteroatom-unsubstituted Cₙ-acyl, as that term is defined above. The group, —NHCO₂CH₃, is a non-limiting example of a heteroatom-substituted amido group.

The term “alkylthio” includes straight-chain alkylthio, branched-chain alkylthio, cycloalkylthio, cyclic alkylthio, heteroatom-unsubstituted alkylthio, heteroatom-substituted alkylthio, heteroatom-unsubstituted Cₙ-alkylthio, and heteroatom-substituted Cₙ-alkylthio. The term “heteroatom-unsubstituted Cₙ-alkylthio” refers to a group, having the structure —SR, in which R is a heteroatom-unsubstituted Cₙ-alkyl, as that term is defined above. The group, —SCH₃, is an example of a heteroatom-unsubstituted alkylthio group. The term “heteroatom-substituted Cₙ-alkylthio” refers to a group, having the structure —SR, in which R is a heteroatom-substituted Cₙ-alkyl, as that term is defined above.

The term “alkenylthio” includes straight-chain alkenylthio, branched-chain alkenylthio, cycloalkenylthio, cyclic alkenylthio, heteroatom-unsubstituted alkenylthio, heteroatom-substituted alkenylthio, heteroatom-unsubstituted Cₙ-alkenylthio, and heteroatom-substituted Cₙ-alkenylthio. The term “heteroatom-unsubstituted Cₙ-alkenylthio” refers to a group, having the structure —SR, in which R is a heteroatom-unsubstituted Cₙ-alkenyl, as that term is defined above. The term “heteroatom-substituted Cₙ-alkenylthio” refers to a group, having the structure —SR, in which R is a heteroatom-substituted Cₙ-alkenyl, as that term is defined above.

The term “alkynylthio” includes straight-chain alkynylthio, branched-chain alkynylthio, cycloalkynylthio, cyclic alkynylthio, heteroatom-unsubstituted alkynylthio, heteroatom-substituted alkynylthio, heteroatom-unsubstituted Cₙ-alkynylthio, and heteroatom-substituted Cₙ-alkynylthio. The term “heteroatom-unsubstituted Cₙ-alkynylthio” refers to a group, having the structure —SR, in which R is a heteroatom-unsubstituted Cₙ-alkynyl, as that term is defined above. The term “heteroatom-substituted Cₙ-alkynylthio” refers to a group, having the structure —SR, in which R is a heteroatom-substituted Cₙ-alkynyl, as that term is defined above.

The term “arylthio” includes heteroatom-unsubstituted arylthio, heteroatom-substituted arylthio, heteroatom-unsubstituted Cₙ-arylthio, heteroatom-substituted Cₙ-arylthio, heteroarylthio, and heterocyclic arylthio groups. The term “heteroatom-unsubstituted Cₙ-arylthio” refers to a group, having the structure —SAr, in which Ar is a heteroatom-unsubstituted Cₙ-aryl, as that term is defined above. The group, —SC₆H₅, is an example of a heteroatom-unsubstituted arylthio group. The term “heteroatom-substituted Cₙ-arylthio” refers to a group, having the structure —SAr, in which Ar is a heteroatom-substituted Cₙ-aryl, as that term is defined above.

The term “aralkylthio” includes heteroatom-unsubstituted aralkylthio, heteroatom-substituted aralkylthio, heteroatom-unsubstituted Cₙ-aralkylthio, heteroatom-substituted Cₙ-aralkylthio, heteroaralkylthio, and heterocyclic aralkylthio groups. The term “heteroatom-unsubstituted Cₙ-aralkylthio” refers to a group, having the structure —SAr, in which Ar is a heteroatom-unsubstituted Cₙ-aralkyl, as that term is defined above. The group, —SCH₂C₆H₅, is an example of a heteroatom-unsubstituted aralkyl group. The term “heteroatom-substituted Cₙ-aralkylthio” refers to a group, having the structure —SAr, in which Ar is a heteroatom-substituted Cₙ-aralkyl, as that term is defined above.

The term “acylthio” includes straight-chain acylthio, branched-chain acylthio, cycloacylthio, cyclic acylthio, heteroatom-unsubstituted acylthio, heteroatom-substituted acylthio, heteroatom-unsubstituted Cₙ-acylthio, heteroatom-substituted Cₙ-acylthio, alkylcarbonyloxy, arylcarbonyloxy, alkoxycarbonyloxy, aryloxycarbonyloxy, and carboxylate groups. The term “heteroatom-unsubstituted Cₙ-acylthio” refers to a group, having the structure —SAc, in which Ac is a heteroatom-unsubstituted Cₙ-acyl, as that term is defined above. The group, —SCOCH₃, is an example of a heteroatom-unsubstituted acylthio group. The term “heteroatom-substituted Cₐ-acylthio” refers to a group, having the structure —SAc, in which Ac is a heteroatom-substituted Cₙ-acyl, as that term is defined above.

The term “alkylsilyl” includes straight-chain alkylsilyl, branched-chain alkylsilyl, cycloalkylsilyl, cyclic alkylsilyl, heteroatom-unsubstituted alkylsilyl, heteroatom-substituted alkylsilyl, heteroatom-unsubstituted Cₙ-alkylsilyl, and heteroatom-substituted Cₙ-alkylsilyl. The term “heteroatom-unsubstituted Cₙ-alkylsilyl” refers to a radical, having a single silicon atom as the point of attachment, further having one, two, or three saturated carbon atoms attached to the silicon atom, further having a linear or branched, cyclic or acyclic structure, containing a total of n carbon atoms, all of which are nonaromatic, 5 or more hydrogen atoms, a total of 1 silicon atom, and no additional heteroatoms. For example, a heteroatom-unsubstituted C₁-C₁₀-alkylsilyl has 1 to 10 carbon atoms. An alkylsilyl group includes dialkylamino groups. The groups, —Si(CH₃)₃ and —Si(CH₃)₂C(CH₃)₃, are non-limiting examples of heteroatom-unsubstituted alkylsilyl groups. The term “heteroatom-substituted Cₙ-alkylsilyl” refers to a radical, having a single silicon atom as the point of attachment, further having at least one, two, or three saturated carbon atoms attached to the silicon atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the silicon atom at the point of attachment, wherein each additional heteroatom is independently selected from the group consisting of N, O, F, Cl, Br, I, Si, P, and S. For example, a heteroatom-substituted C₁-C₁₀-alkylsilyl has 1 to 10 carbon atoms.

The term “phosphonate” includes straight-chain phosphonate, branched-chain phosphonate, cyclophosphonate, cyclic phosphonate, heteroatom-unsubstituted phosphonate, heteroatom-substituted phosphonate, heteroatom-unsubstituted Cₙ-phosphonate, and heteroatom-substituted Cₙ-phosphonate. The term “heteroatom-unsubstituted Cₙ-phosphonate” refers to a radical, having a single phosphorous atom as the point of attachment, further having a linear or branched, cyclic or acyclic structure, further having a total of n carbon atoms, 2 or more hydrogen atoms, a total of three oxygen atom, and no additional heteroatoms. The three oxygen atoms are directly attached to the phosphorous atom, with one of these oxygen atoms doubly bonded to the phosphorous atom. For example, a heteroatom-unsubstituted C₀-C₁₀-phosphonate has 0 to 10 carbon atoms. The groups, —P(O)(OH)₂, —P(O)(OH)OCH₃, —P(O)(OH)OCH₂CH₃, —P(O)(OCH₃)₂, and —P(O)(OH)(OC₆H₅) are non-limiting examples of heteroatom-unsubstituted phosphonate groups. The term “heteroatom-substituted Cₙ-phosphonate” refers to a radical, having a single phosphorous atom as the point of attachment, further having a linear or branched, cyclic or acyclic structure, further having a total of n carbon atoms, 2 or more hydrogen atoms, three or more oxygen atoms, three of which are directly attached to the phosphorous atom, with one of these three oxygen atoms doubly bonded to the phosphorous atom, and further having at least one additional heteroatom in addition to the three oxygen atoms, wherein each additional heteroatom is independently selected from the group consisting of N, O, F, Cl, Br, I, Si, P, and S. For example, a heteroatom-unsubstituted C₀-C₁₀-phosphonate has 0 to 10 carbon atoms.

The term “phosphinate” includes straight-chain phosphinate, branched-chain phosphinate, cyclophosphinate, cyclic phosphinate, heteroatom-unsubstituted phosphinate, heteroatom-substituted phosphinate, heteroatom-unsubstituted Cₙ-phosphinate, and heteroatom-substituted Cₙ-phosphinate. The term “heteroatom-unsubstituted Cₙ-phosphinate” refers to a radical, having a single phosphorous atom as the point of attachment, further having a linear or branched, cyclic or acyclic structure, further having a total of n carbon atoms, 2 or more hydrogen atoms, a total of two oxygen atom, and no additional heteroatoms. The two oxygen atoms are directly attached to the phosphorous atom, with one of these oxygen atoms doubly bonded to the phosphorous atom. For example, a heteroatom-unsubstituted C₀-C₁₀-phosphinate has 0 to 10 carbon atoms. The groups, —P(O)(OH)H, —P(O)(OH)CH₃, —P(O)(OH)CH₂CH₃, —P(O)(OCH₃)CH₃, and —P(O)(OC₆H₅)H are non-limiting examples of heteroatom-unsubstituted phosphinate groups. The term “heteroatom-substituted Cₙ-phosphinate” refers to a radical, having a single phosphorous atom as the point of attachment, further having a linear or branched, cyclic or acyclic structure, further having a total of n carbon atoms, 2 or more hydrogen atoms, two or more oxygen atoms, two of which are directly attached to the phosphorous atom, with one of these two oxygen atoms doubly bonded to the phosphorous atom, and further having at least one additional heteroatom in addition to the two oxygen atoms, wherein each additional heteroatom is independently selected from the group consisting of N, O, F, Cl, Br, I, Si, P, and S. For example, a heteroatom-unsubstituted C₀-C₁₀-phosphinate has 0 to 10 carbon atoms.

Any apparently unfulfilled valency is to be understood to be properly filled by hydrogen atom(s). For example, a compound with a substituent of —O or —N is to be understood to be —OH or —NH₂, respectively.

Any genus, subgenus, or specific compound discussed herein is specifically contemplated as being excluded from any embodiment described herein.

Compounds described herein may be prepared synthetically using conventional organic chemistry methods known to those of skill in the art and/or are commercially available (e.g., ChemBridge Co., San Diego, CA).

The disclosure is also intended to encompass salts of any of the compounds of the present disclosure. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like. A salt may be a pharmaceutically acceptable salt, for example. Thus, pharmaceutically acceptable salts of compounds of the present disclosure are contemplated.

The term “pharmaceutically acceptable salts,” as used herein, refers to salts of compounds of this disclosure that are substantially non-toxic to living organisms. Typical pharmaceutically acceptable salts include those salts prepared by reaction of a compound of this disclosure with an inorganic or organic acid, or an organic base, depending on the substituents present on the compounds of the disclosure.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like.

Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like.

Suitable pharmaceutically acceptable salts may also be formed by reacting the agents of the disclosure with an organic base such as methylamine, ethylamine, ethanolamine, lysine, ornithine and the like.

Pharmaceutically acceptable salts include the salts formed between carboxylate or sulfonate groups found on some of the compounds of this disclosure and inorganic cations, such as sodium, potassium, ammonium, or calcium, or such organic cations as isopropylammonium, trimethylammonium, tetramethylammonium, and imidazolium.

Derivatives of compounds of the present disclosure are also contemplated. In certain aspects, “derivative” refers to a chemically modified compound that still retains the desired effects of the compound prior to the chemical modification. Such derivatives may have the addition, removal, or substitution of one or more chemical moieties on the parent molecule. Non-limiting examples of the types modifications that can be made to the compounds and structures disclosed herein include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, heteroatoms such as N, S, or O can be substituted into the structure instead of a carbon atom.

Compounds employed in methods of the disclosure may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S- or the R-configuration, as defined by the IUPAC 1974 Recommendations. Compounds may be of the D- or L- form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

As noted above, compounds of the present disclosure may exist in prodrug form. As used herein, “prodrug” is intended to include any covalently bonded carriers which release the active parent drug or compounds that are metabolized in vivo to an active drug or other compounds employed in the methods of the disclosure in vivo when such prodrug is administered to a subject. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a subject, cleaves to form a free hydroxyl, free amino, or carboxylic acid, respectively. Other examples include, but are not limited to, acetate, formate, and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (2002), which is incorporated herein by reference.

### C. PHARMACEUTICAL FORMULATIONS AND ADMINISTRATION THEREOF

Pharmaceutical compositions of the present disclosure comprise an effective amount of one or more candidate substance or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The compounds of the disclosure may comprise different types of carriers depending on whether it is to be administered in solid, liquid or aerosol form, and whether it need to be sterile for such routes of administration as injection. The present disclosure can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, systemically, subcutaneously, subconjunctival, intravesicularlly, mucosally, intrapericardially, intraumbilically, intraocularally, orally, locally, via inhalation (e.g., aerosol inhalation), via injection, via infusion, via continuous infusion, via localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 1990).

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% by weight or volume of a compound of the present disclosure. In other embodiments, the compound may comprise between about 2% to about 75% of the weight or volume of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain aspects, the amount of compound administered to a subject results in a biological concentration of between approximately 0.001 to 1,000 nM or μM, or any range derivable therein, of the compound. In certain aspects, the amount of compound administered to a subject results in a biological concentration of approximately, at least, or at most 0.001, 0.01, 0.1, 1, 10, 100, or 1,000 nM or μM of the compound. The biological concentration may be a concentration present in a biological sample, such as a blood sample, saliva sample, urine sample, fecal sample, and/or biopsy. The biological concentration may be a concentration at a site of interest, such as a tumor site, or a tissue of interest. In certain aspects, the amount of compound provided to a cell results in a concentration of between approximately 0.001 to 1,000 nM or VM, or any range derivable therein, of the compound. In certain aspects, the amount of compound provided to a cell results in a concentration of approximately, at least, or at most 0.001, 0.01, 0.1, 1, 10, 100, or 1,000 nM or μM of the compound.

In certain aspects, the compounds disclosed herein are administered or provided in a set dosing regimen. The dosing regimen of the compounds disclosed herein may be determined by one skilled in the art and can be tailored to the subject receiving the compounds. In certain aspects, the dosing regimen comprises administering or providing the compound once. In certain aspects, the dosing regimen comprises administering or providing the compound more than once, including 2, 3, 4, 5, 6, 7, 8, 9, 10, or more, or any range derivable therein, times. In certain aspects, the compound is provided or administered several times a day, every day, every week, every month, or every year.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The candidate substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine, or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. It may be preferable to include isotonic agents, such as, for example, sugars, sodium chloride or combinations thereof.

In other embodiments, one may use eye drops, nasal solutions or sprays, aerosols or inhalants. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in certain embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the candidate substance is prepared for administration by such routes as oral ingestion. In these embodiments, the composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. In certain embodiments, carriers for oral administration comprise inert diluents, assimilable edible carriers or combinations thereof. In other aspects of the disclosure, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, or both.

Additional formulations which are suitable for other modes of administration include suppositories. Suppositories are solid dosage forms of various weights and shapes, usually medicated, for insertion into the rectum, vagina, or urethra. After insertion, suppositories soften, melt or dissolve in the cavity fluids. In general, for suppositories, traditional carriers may include, for example, polyalkylene glycols, triglycerides, or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, certain methods of preparation may include vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin, or combinations thereof.

### D. ORGANISMS AND CELL SOURCE

Cells that may be used in some methods can be from a variety of sources. Embodiments include the use of mammalian cells, such as cells from monkeys, chimpanzees, rabbits, mice, rats, ferrets, dogs, pigs, humans, and cows. Alternatively, the cells may be from fruit flies, yeast, or E. coli.

Methods can involve cells, tissues, or organs involving the heart, lung, kidney, liver, bone marrow, pancreas, skin, bone, vein, artery, cornea, blood, small intestine, large intestine, brain, spinal cord, smooth muscle, skeletal muscle, ovary, testis, uterus, and umbilical cord.

Moreover, methods can be employed in cells of the following type: platelet, myelocyte, erythrocyte, lymphocyte, adipocyte, fibroblast, epithelial cell, endothelial cell, smooth muscle cell, skeletal muscle cell, endocrine cell, glial cell, neuron, secretory cell, barrier function cell, contractile cell, absorptive cell, mucosal cell, limbus cell (from cornea), stem cell (totipotent, pluripotent or multipotent), unfertilized or fertilized oocyte, or sperm.

Moreover, methods can be implemented with or in plants or parts of plants, including fruit, flowers, leaves, stems, seeds, cuttings. Plants can be agricultural, medicinal, or decorative.

### E. KITS

Kits are also contemplated as being used in certain aspects of the present disclosure. For instance, a compound or composition of the present disclosure can be included in a kit. The kit may further include other materials desirable from a commercial and user standpoint, including buffers, diluents, filters, needles, syringes, and package inserts with instructions. Instructions can include an explanation of how to administer, apply, use, and/or maintain the compositions.

A kit can include a container. Containers can include a bottle, a vial, a tube, a flask, a bag, a syringe, a metal tube, a laminate tube, a plastic tube, a dispenser, a pressurized container, a barrier container, a package, a compartment, or other types of containers such as injection or blow-molded plastic containers into which the compositions or desired bottles, dispensers, or packages are retained. Where there are multiple components in the kit, the kit also may contain a second, third, or other additional container into which the additional components may be separately placed. The container(s) may be formed from a variety of materials such as glass, plastic (such as polyvinyl chloride or polyolefin), or metal alloy (such as stainless steel or a nickel-molybdenum alloy). The kit and/or container(s) can include indicia on its surface. The indicia, for example, can be a word, a phrase, an abbreviation, a picture, or a symbol.

The container(s) can dispense a pre-determined amount of a composition. In other embodiments, the container(s) can be squeezed (e.g., metal, laminate, or plastic tube) to dispense a desired amount of the composition. The composition can be dispensed as, e.g., a tablet, a spray, a foam, an aerosol, a liquid, a fluid, or a semi-solid. The container(s) can have spray, pump, or squeeze mechanisms.

### F. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

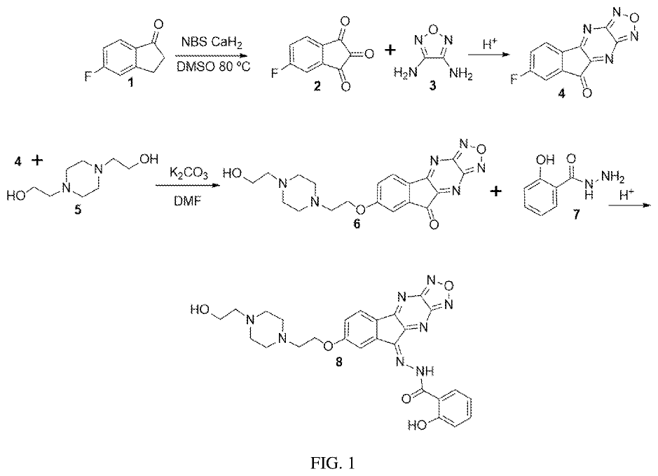

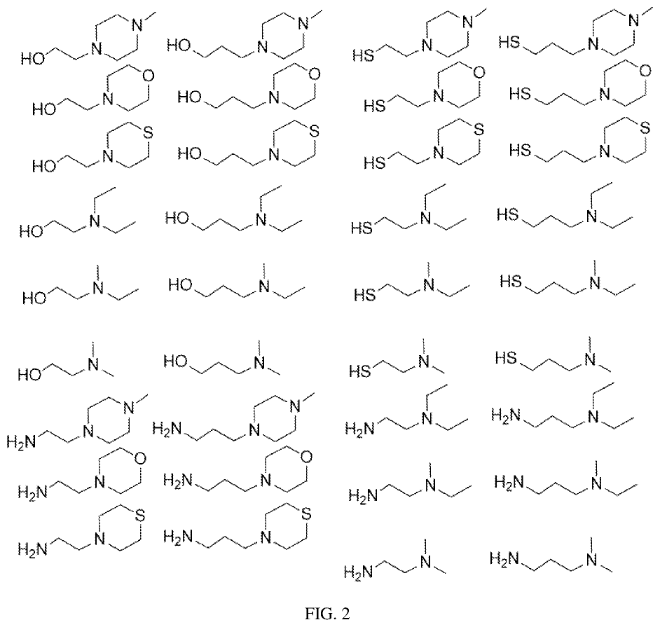

Referring to FIG. 1, 6-fluoroindanone 1 is reacted with N-bromosuccinimide and calcium hydride to produce 6-fluoroindane-1,2,3-trione 2. (Synthetic Communications 28, 859 (1998)). Acid-catalyzed dehydrative coupling between 2 and 1,2,5-oxadiazole-3,4-diamine 3 produces 7-fluoro-9-oxo-9H-2-oxa-1,3,4,10-tetreaaza-cyclopenta[b]fluorene 4. (WO 2001/107209, J Med Chem 58, 4678 (2015)). Etherification of oxadiazole pyrazinone 4 with N,N-bis-(2-hydroxyethyl)piperazine 5 produces aryl ether compound 6. Acid-catalyzed dehydrative coupling between aryl ether 6 and 2-hydroxybenzohydrazide 7 produces benzohydrazide compound 8. (WO 2016/187050). FIG. 2 depicts an exemplary, but not limiting, series of nucleophilic hydroxyl, sulfhydryl, and amine compounds that can be reacted with oxadiazole pyrazinone 4 (and related compounds) listed above to produce aryl ether, aryl thioether, and aryl amine variants of benzohydrazide compound 8.

### Example 2

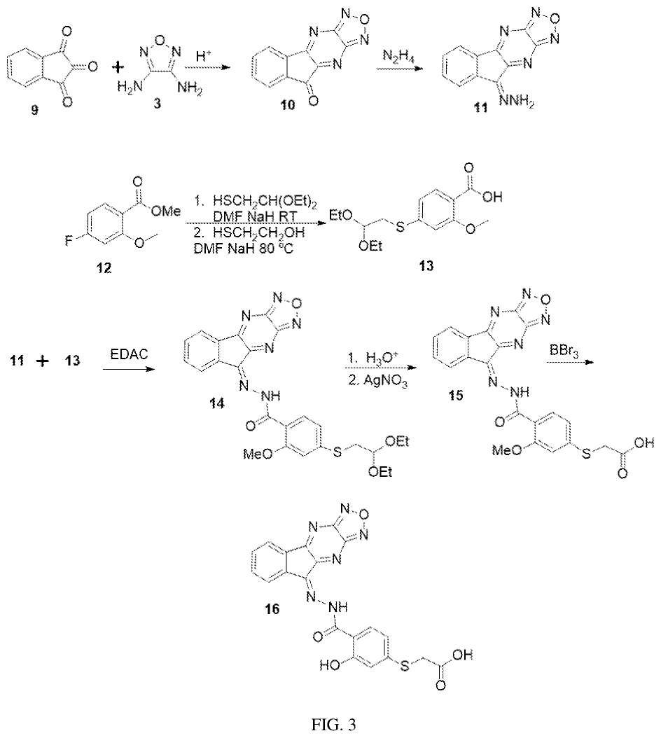

Referring to FIG. 3, acid-catalyzed dehydrative coupling between indane-1,2,3-trione 9 and 1,2,5-oxadiazole-3,4-diamine 3 produces 9-oxo-9H-2-oxa-1,3,4,10-tetreaaza-cyclopenta[b]fluorene 10. Reaction between hydrazine and oxadiazole pyrazinone 10 gives rise to hydrazone 11. Methyl 4-fluoro-2-methoxybenzoate 12 is reacted with 2,2-diethoxyethane-1-thiol followed by ethanol-2-thiol to produce diethoxyethylthio-benzoic acid 13. Reaction between hydrazone 11 and diethoxyethylthio-benzoic acid 13 produces diethoxy benzohydrazide 14. The diethoxy moiety on compound 14 is hydrolyzed to the corresponding aldehyde and subsequently oxidized to carboxylic acid 15. BBr₃ is then used to demethylate the aromatic methyl ether to produce phenolic compound 16. Alternatively acid 13 can be reacted with hydrazine to form an acyl hydrazine, which can then be coupled with ketone 10, to form Intermediate 14.

### Example 3

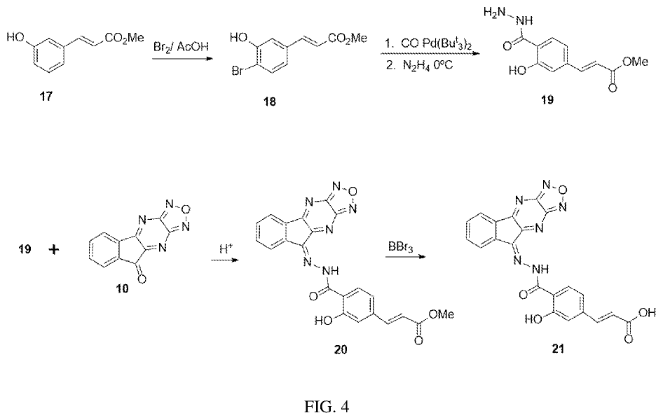

Referring to FIG. 4, methyl (E)-3-(3-hydroxyphenyl)acrylate 17 is brominated using bromine/acetic acid to produce bromophenol 18. (J Med Chem 62, 3590 (2019)). Carbonylation of the bromophenol followed by treatment with hydrazine gives benzohydrazide 19. Condensation between benzohydrazide 19 and the carbonyl of oxadiazole pyrazinone 10 produces hydrazone 20, which is subsequently demethylated using BBr₃ to produce compound 21. Alternatively, compound 18 can be carbonylated as described, but the acyl Pd intermediate can be trapped by hydroxide to give the carboxylic acid corresponding to 19. This can then be reacted with hydrazone 11, to form intermediate 20.

### Example 4

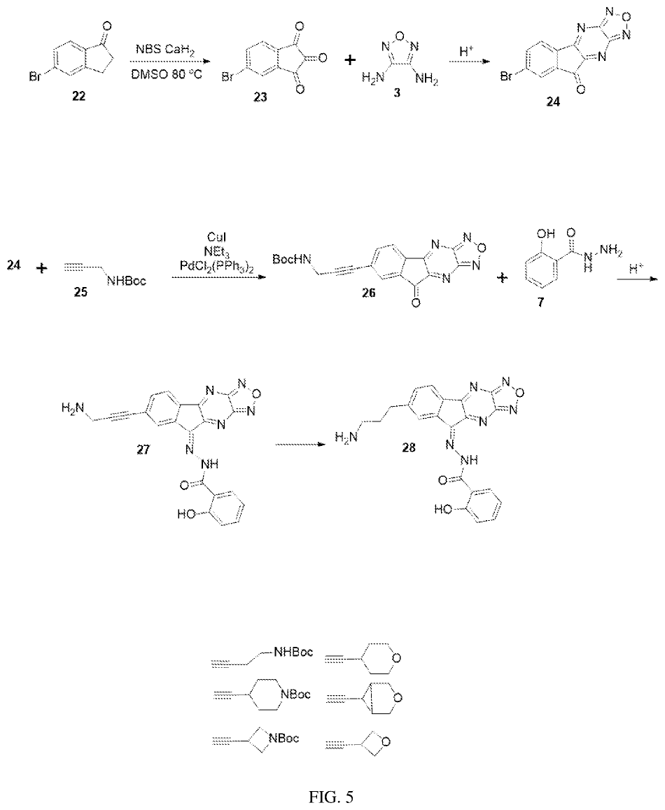

Referring to FIG. 5, bromoindanone 22 is reacted with N-bromosuccinamide and calcium hydride to produce indane-trione 23. (Synthetic Communications 28, 859 (1998)). Acid-catalyzed dehydrative coupling between indane-trione 23 and 1,2,5-oxadiazole-3,4-diamine 3 produces 7-bromo-9-oxo-9H-2-oxa-1,3,4,10-tetreaaza-cyclopenta[b]fluorene 24. Alternatively, 24 can be prepared by direct bromination of 10. (WO2001/079209) Sonogashira alkynylination of 24 with N-Boc propargylamine 25 produces aryl acetylene 26. Hydrazone formation between aryl acetylene 26 and 2-hydroxybenzohydrazide 7 gives benzohydrazide 27. The benzohydrazide 27 alkyne group can be selectively reduced to compound 28. Alternatively, ketone 26 could be converted to the corresponding hydrazone and that acylated with salicylic acid to give the N-Boc version of 27, which could be deprotected by acid to give 27. At the end of the reaction scheme in FIG. 5 is an exemplary, but not exclusive, series of terminal acetylenic compounds that can be coupled to oxadiazole pyrazinone 24 and related aryl halide compounds.

### Example 5

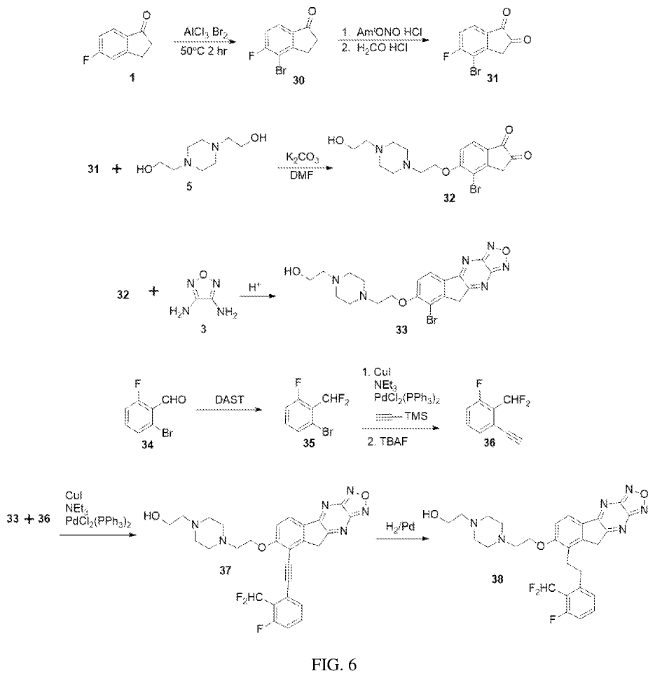

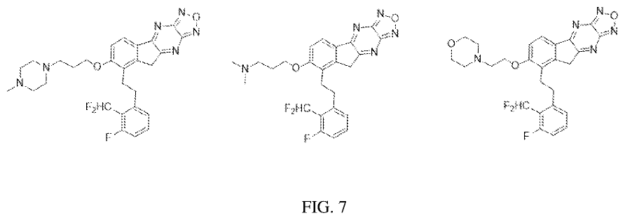

Referring to FIG. 6, aluminum chloride-catalyzed bromination of fluoroindanone 1 produces fluoro bromoindanone 30. (WO 2007/050732) Oxidation of fluoro bromoindanone 30 produces indanedione 31. (WO 1998/020344, EP 1749822) Etherification of indanedione 3 with bis-ethanol piperazine 5 produces aryl ether compound 32. Any of the compounds depicted in FIG. 2 can be used in place of bis-ethanol piperazine 5 to produce related derivatives. Reaction between aryl ether compound 32, and acid-catalyzed reaction of 32 with 1,2,5-oxadiazole-3,4-diamine 3 gives 7-aminoalkoxy-8-bromo-9H-2-oxa-1,3,4,10-tetreaaza-cyclopenta[b]fluorene 33. 2-bromo-6-fluorobenzaldehyde 34 is fluorinated using diethylaminosulfur trifluoride (DAST) to produce aryl halide 35. Sonogashira coupling of 35 with TMS acetylene, followed by TBAF-mediated removal of the TMS group, produces terminal acetylenic compound 36. A second Sonogashira coupling between 9H-2-oxa-1,3,4,10-tetreaaza-cyclopenta[b]fluorene 33 and terminal acetylenic compound 36 produces diphenyl acetylene compound 37, which is subsequently reduced catalytically, preferably with hydrogen and palladium, to provide compound 38. FIG. 7 depicts an exemplary, but not exclusive, series of analogues of compound 38 that are produced using compounds from FIG. 2.

### Example 6

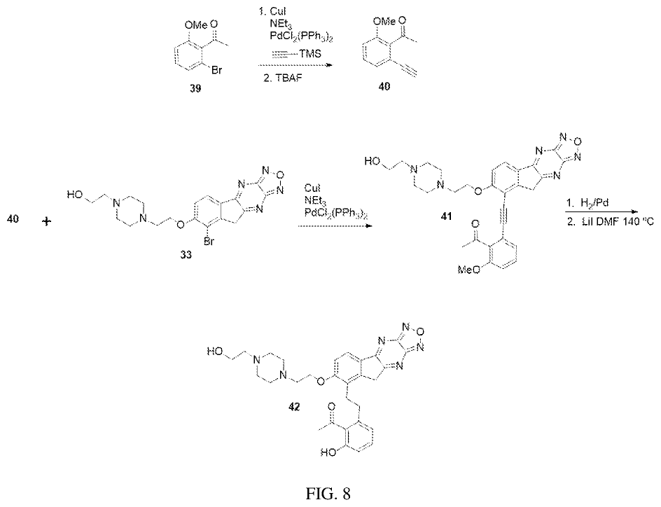

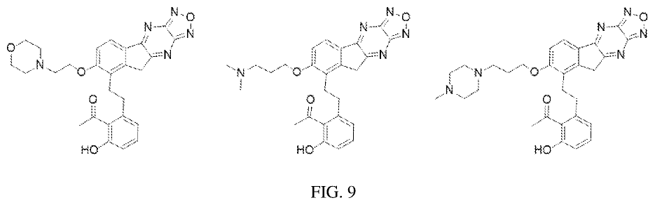

FIG. 8 depicts a scheme for producing different 8-(2-arylethyl) derivatives of 7-aminoalkoxy-9H-2-oxa-1,3,4,10-tetreaaza-cyclopenta[b]fluorenes. Sonogashira coupling between TMS acetylene 1-(2-bromo-6-methoxyphenyl)ethan-1-one 39 followed by TBAF-mediated removal of the TMS group produces 1-(2-ethynyl-6-methoxyphenyl)ethan-1-one 40. A second Sonogashira coupling between aryl halide 33 and 1-(2-ethynyl-6-methoxyphenyl)ethan-1-one 40 produces diaryl acetylene compound 41, which is subsequently reduced to provide compound 42. FIG. 9 depicts an exemplary, but not exclusive, series of analogues of compound 42 that are produced using 7-position substituents from FIG. 2.

### Example 7

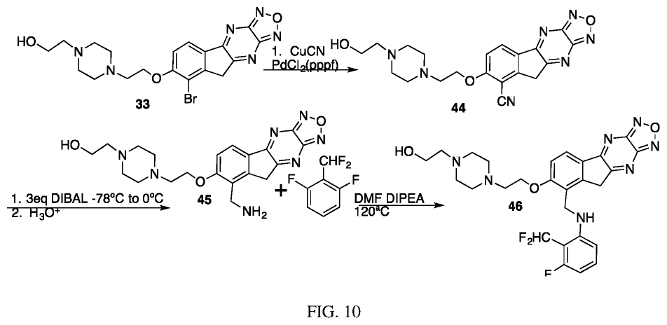

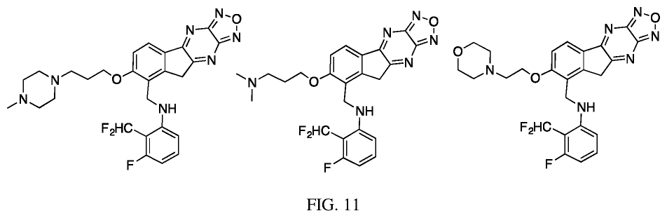

FIG. 10 depicts a scheme for producing different 8-(N-arylmethylamino) derivatives of 7-aminoalkoxy-9H-2-oxa-1,3,4,10-tetreaaza-cyclopenta[b]fluorenes. Bromide 33 is cyanated under standard conditions with CuCN and PdCl₂(dppf) to give nitrile 44. This is fully reduced to the corresponding aminomethyl derivative 45 with excess DIBAL-H, and the newly formed amine then displaces a reactive fluorine in 2-difluoromethyl-1,3-difluorobenzene to give compound 46. FIG. 11 depicts an exemplary, but not exclusive, series of analogues of compound 46 that are produced using 7-position substituents from FIG. 2.

### Example 8

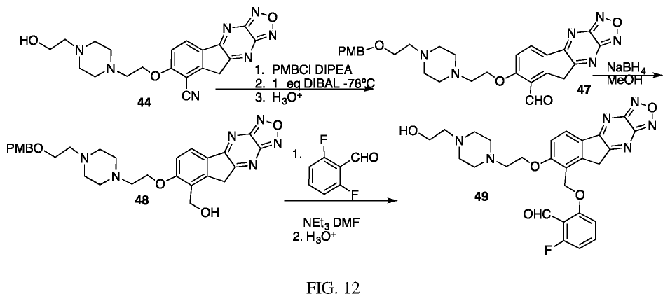

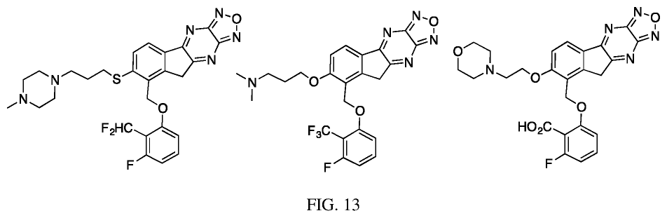

FIG. 12 depicts a scheme for producing different 8-(aryloxymethyl) derivatives of 7-aminoalkoxy-9H-2-oxa-1,3,4,10-tetreaaza-cyclopenta[b]fluorenes. Nitrile 44 is hydroxyl protected with p-methoxybenzyl chloride to form a PMB ether, and then partially reduced to the corresponding imine with DIBAL-H at −78° C., and then hydrolyzed to the corresponding aldehyde derivative 47, which is further reduced with NaBH₄ to the hydroxymethyl compound 48. The newly formed alcohol then displaces a reactive fluorine in 1,3-difluorobenzaldehyde, and the PMB group is removed under acidic conditions to give compound 49. FIG. 13 depicts an exemplary, but not exclusive, series of analogues of compound 49 that are produced using 7-position substituents from FIG. 2.

### Example 9

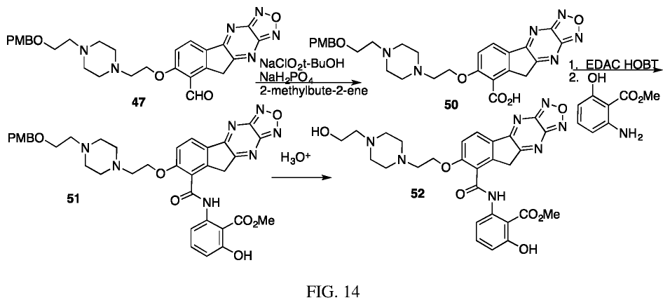

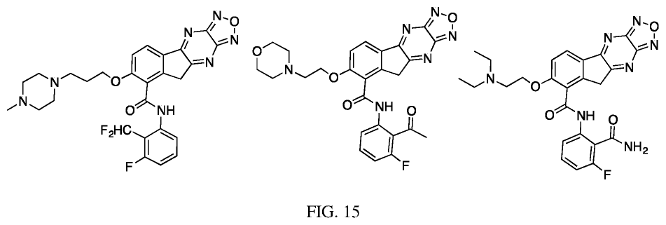

FIG. 14 depicts a scheme for producing different 8-(N-arylcarboxamido) derivatives of 7-aminoalkoxy-9H-2-oxa-1,3,4,10-tetreaaza-cyclopenta[b]fluorenes. Aldehyde 47 is Pinnock oxidized (NaClO2, t-butanol, NaH2PO4, 2-methylbut-2-ene) to the corresponding carboxylic acid 50. The corresponding HOBT ester is formed under standard conditions and then reacted with commercially available 6-hydroxyanthranilic acid to give the amide 51, which is then deprotected under standard conditions to give 52. FIG. 15 depicts an exemplary, but not exclusive, series of analogues of compound 52 that are produced using 7-position substituents from FIG. 2.

### Example 10

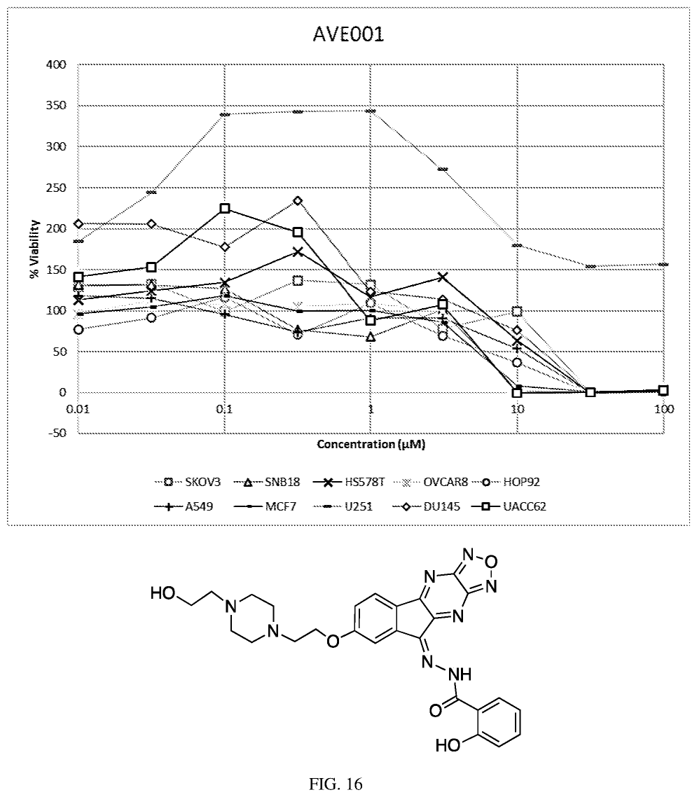

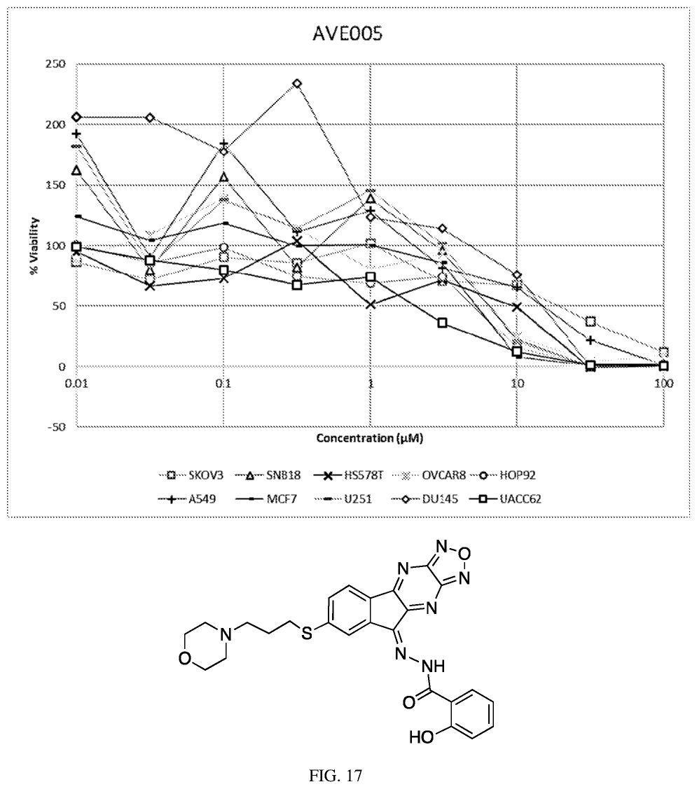

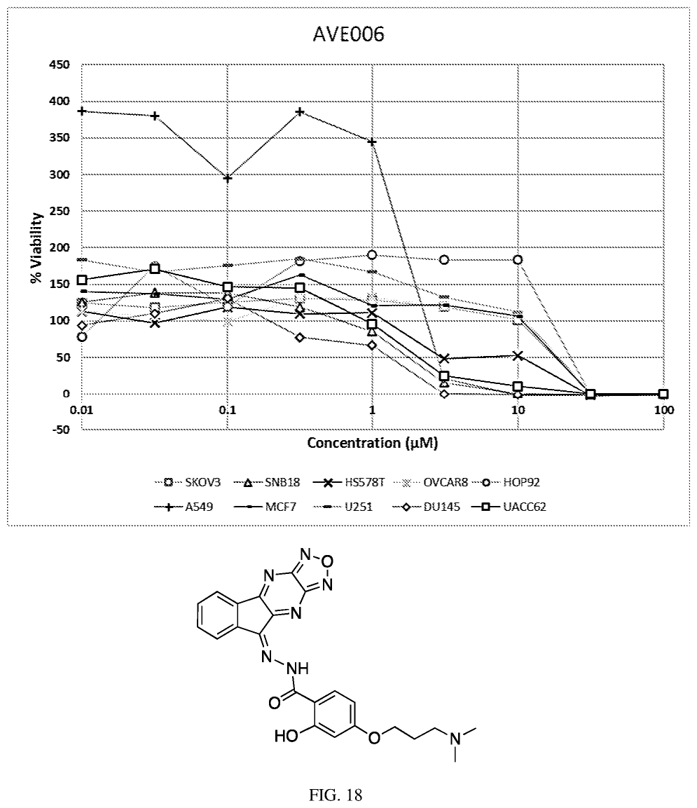

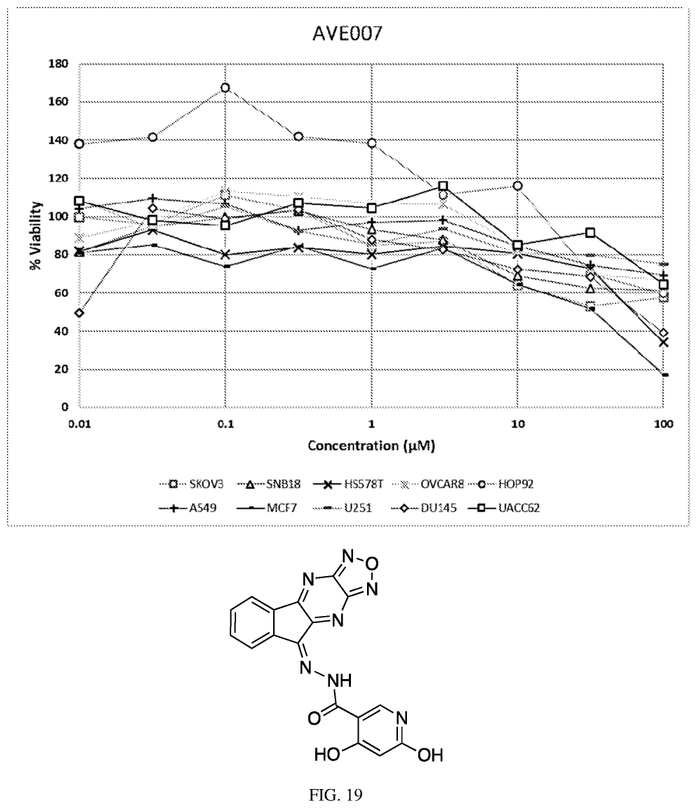

FIGS. 16-19 show cell lines treated with exemplary RAL-interacting protein inhibitors at the indicated concentrations and assayed via MTT assay. Briefly, cells were cultured in a 96 well plate during the log growth period. The cells were plated at 4×10³ cells/well. 24 hours after plating, the inhibitors or a vehicle control were added to the cells. 96 hours after adding the drugs, the MTT assay was performed by adding 50 μL of serum-free media and 50 μL of MTT reagent to each well. Absorbance was read at OD=590 nm, which is proportional to the cell number. The percent toxicity was calculated by the following equation, where the control is the culture medium background reading:

$$\% Cytotoxicity = \frac{( 100 \times ( Control - Sample ) )}{Control}$$

All of the methods and apparatuses disclosed and claimed herein can be made and executed without undue experimentation in light of the present disclosure. While the compositions and methods of this invention have been described in terms of preferred embodiments, it will be apparent to those of skill in the art that variations may be applied to the methods and apparatuses and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of treating or preventing cancer in a subject, the method comprising: administering to the subject an effective amount of a compound of formula (I): 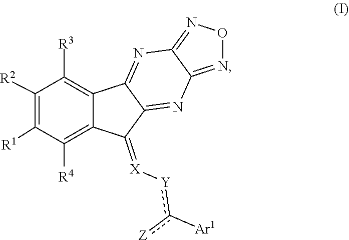
wherein
X is N or CH;
Y is NH or O when Z is a carbonyl oxygen, or CH when Z is F;
Z is ═O or F;
R¹ is H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-Lt)ₚ, (C₆₋₁₀ bridged bicycloheteroalkyl-C₀₋₄ alkylene-Lt)ₚ, a substituted or unsubstituted heterocycloalkoxy, or a substituted or unsubstituted heterocyclosulfide;
Ar¹ is 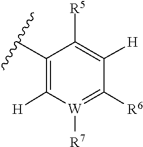
W is N or C;
R² and R⁴ are each independently H, halogen, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkyl-L¹)ₚ, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
R³ is H, halogen, or C₁₋₆ lower alkyl;
R⁵ is NH₂, OH or F;
R⁶ is H, OH, F, Cl, Br, CH₃, ethynyl, cyano, a substituted or unsubstituted alkenyl, a substituted or unsubstituted alkoxy, or a substituted or unsubstituted sulfide;
R⁷ is nothing when W is N, or H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ when W is C;
R⁹ and R¹⁰ are each independently H, R³, or R⁹ and R¹⁰, and the N atom that connects R⁹ and R¹⁰ can form a heterocyclic ring of 4 to 8 members which can further comprise N, O, S, or SO₂;
R¹¹ is C₁₋₆ lower alkyl, C₂₋₆ lower alkenyl, C₂₋₆ lower alkynyl, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
L¹ is independently at each occurrence a bond, O, NH, NR⁴, N(C₁₋₆ lower acyl), S, SO, SO₂, CO, CONR⁴, CO₂, NR⁴CO, OCONH, OCONR⁴, OCO₂, NR⁴CONR⁴, NR⁴CSNR⁴, NR⁴CO, OCO, SO₂NR⁴, or NR⁴SO₂;
p is 1-3, wherein each repeat unit can be the same or different with respect to both cyclic and linear elements; and
wherein each of R¹—R¹¹, independently, can optionally be substituted with up to 3 substituents selected from the group consisting of OH, CN, C₁₋₆ lower alkyl, C₁₋₆ lower alkoxy, C₁₋₆ lower alkylthio, C₁₋₆ lower alkylamino, C₁₋₆ lower dialkylamino, C₁₋₆ lower acyloxy, optionally substituted phenyl, optionally substituted C₅₋₆ heteroaryl, halogen, CF₃, OCF₃, oxo, (OCH₂CH₂O)₁₋₁₀OH, and (OCH₂CH₂O)₁₋₁₀OCH₃;
or an enantiomer, diastereoisomer, racemic mixture, or salt(s) thereof.

2. The method of claim 1, wherein any hydrogen or carbon present in the compound are replaced by a corresponding isotope ²H, ³H, ¹³C or ¹⁴C.

3. The method of claim 1 or 2, wherein the compound of formula (I) inhibits a RAL-interacting protein.

4. The method of any of claims 1-3, wherein the RAL-interacting protein is RLIP76.

5. The method of any of claims 1-5, wherein the compound of formula (I) binds to a transporter substrate binding site of RLIP76.

6. The method of any of claims 1-5, wherein administration of the compound of formula (I) overcomes deleterious effects of p53 gene loss.

7. The method of any of claims 1-6, wherein the subject has cancer, is suspected of having cancer, or has been previously been diagnosed with cancer.

8. The method of any of claims 1-7, wherein the cancer is breast cancer, colon cancer, lung cancer, hepatocellular cancer, pancreatic cancer, prostate cancer, glioblastoma, melanoma, or ovarian cancer.

9. The method of any of claims 1-8, wherein the breast cancer is triple-negative breast cancer, ductal carcinoma in situ, invasive ductal carcinoma, tubular carcinoma of a breast, medullary carcinoma of a breast, mucinous carcinoma of a breast, papillary carcinoma of a breast, cribriform carcinoma of a breast, invasive lobular carcinoma, inflammatory breast cancer, lobular carcinoma in situ, male breast cancer, molecular subtypes of breast cancer, Paget's disease of a nipple, phyllodes tumors of a breast, metastatic breast cancer, or combinations thereof.

10. The method of any of claims 1-9, wherein the compound is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

11. The method of any of claims 1-10, wherein the administration is done prior to, concurrently with, or subsequent to chemotherapy, surgical treatment, or radiation treatment.

12. The method of any of claims 1-11, wherein the compound of formula (I) is administered to the subject at least two, three, four, five, six, seven, eight, nine or ten times.

13. The method of any of claims 1-12, wherein the subject is administered at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, 100, 150, 200, 250, or 300 μg/kg or mg/kg.

14. The method of any of claims 1-13, wherein said subject is further administered a distinct cancer therapy.

15. The method of any of claims 1-14, wherein said distinct cancer therapy comprises surgery, radiotherapy, chemotherapy, toxin therapy, immunotherapy, cryotherapy or gene therapy.

16. The method of any of claims 1-15, wherein the compound of formula (I) is further defined as:

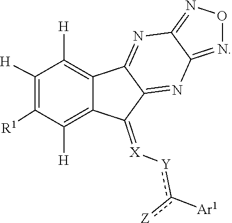

17. The method of any of claims 1-15, wherein the compound of formula (I) is at least one of the following compounds:

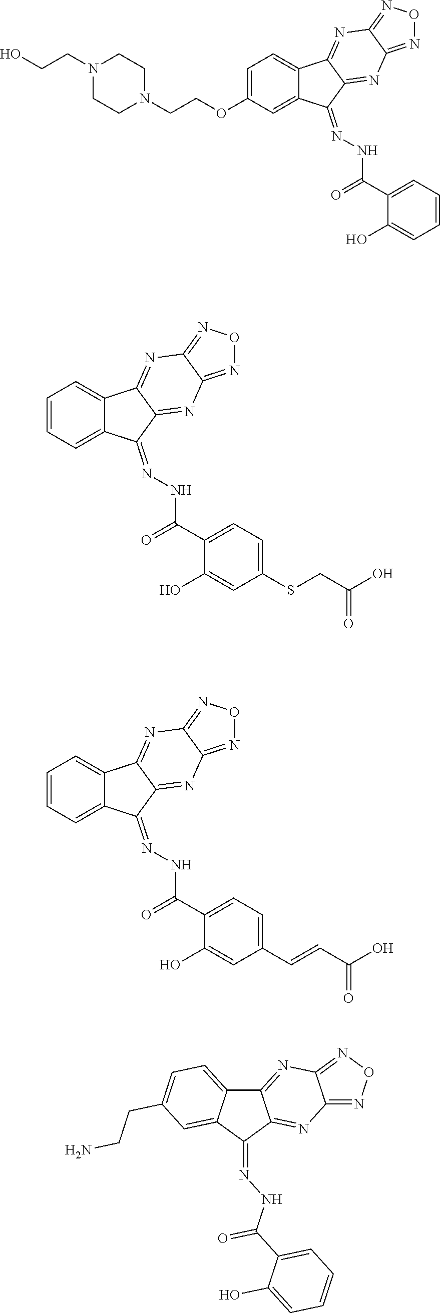 [SMILES: O=C(N/N=C1/c2cc(OCCN3CCN(CCO)CC3)ccc2-c2nc3nonc3nc21)c1ccccc1O ; O=C(O)CSc1ccc(C(=O)N/N=C2/c3ccccc3-c3nc4nonc4nc32)c(O)c1 ; O=C(O)/C=C/c1ccc(C(=O)N/N=C2/c3ccccc3-c3nc4nonc4nc32)c(O)c1 ; NCCc1ccc2c(c1)/C(=N/NC(=O)c1ccccc1O)c1nc3nonc3nc1-2]

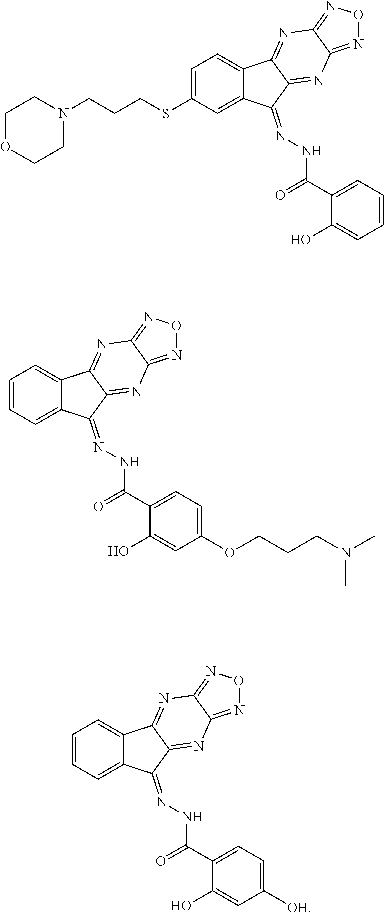 [SMILES: O=C(N/N=C1/c2cc(SCCCN3CCOCC3)ccc2-c2nc3nonc3nc21)c1ccccc1O ; CN(C)CCCOc1ccc(C(=O)N/N=C2/c3ccccc3-c3nc4nonc4nc32)c(O)c1 ; O=C(N/N=C1/c2ccccc2-c2nc3nonc3nc21)c1ccc(O)cc1O]

18. The method of any of claims 1-17, wherein the compound is not

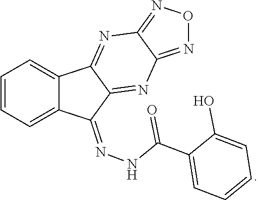 [SMILES: O=C(N/N=C1/c2ccccc2-c2nc3nonc3nc21)c1ccccc1O]

19. The method of any one of claims 1-18, wherein the compound of formula (I) is administered at a dose, or results in a biological concentration of, between approximately 0.001 μM to 1 mM.

20. A method for inhibiting a RAL-interacting protein in a cell comprising providing to the cell an effective amount of a RAL-interacting protein inhibitor, wherein the RAL-interacting protein inhibitor is a compound of the formula (I): 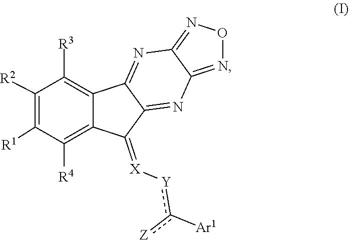
wherein
X is N or CH;
Y is NH or O when Z is a carbonyl oxygen, or CH when Z is F;
Z is ═O or F;
R¹ is H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ, a substituted or unsubstituted heterocycloalkoxy, or a substituted or unsubstituted heterocyclosulfide;
Ar¹ is 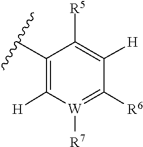
W is N or C;
R² and R⁴ are each independently H, halogen, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkyl-L¹)ₚ, (C₄₋₇ heterocycloalkyl-C₀₋₄ alkylene-L¹)ₚ, (C₆₋₁₀ bridged bicycloheteroalkyl-C₀₋₄ alkylene-L¹)ₚ;
R³ is H, C₁₋₆ lower alkyl;
R⁵ is NH₂, OH or F;
R⁶ is H, OH, F, Cl, Br, CH₃, ethynyl, cyano, a substituted or unsubstituted alkenyl, a substituted or unsubstituted alkoxy, or a substituted or unsubstituted sulfide;
R⁷ is nothing when W is N, or H, OH, OR⁹, OCOR¹¹, OCOR⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo,
cyano, nitro, C₁₋₆ lower alkyl, C₂_lower alkenyl, C₂_lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ when W is C;
R⁹ and R¹⁰ are each independently H, R³, or R⁹ and R¹⁰, and the N atom that connects R⁹ and R¹⁰ can form a heterocyclic ring of 4 to 8 members which can further comprise N, O, S, or SO₂;
R¹¹ is C₁₋₆ lower alkyl, C₂₋₆ lower alkenyl, C₂₋₆ lower alkynyl, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
L¹ is independently at each occurrence a bond, O, NH, NR⁴, N(C₁₋₆ lower acyl), S, SO, SO₂, CO, CONR⁴, CO₂, NR⁴CO, OCONH, OCO NR⁴, OCO₂, NR⁴CONR⁴, NR⁴CSNR⁴, NR⁴CO, OCO, SO₂NR⁴, or NR⁴SO₂,
p is 1-3, wherein each repeat unit can be the same or different with respect to both cyclic and linear elements; and
wherein each of R¹—R¹¹, independently, can optionally be substituted with up to 3 substituents selected from the group consisting of OH, CN, C₁₋₆ lower alkyl, C₁₋₆ lower alkoxy, C₁₋₆ lower alkylthio, C₁₋₆ lower alkylamino, C₁₋₆ lower dialkylamino, C₁₋₆ lower acyloxy, optionally substituted phenyl, optionally substituted C₅₋₆ heteroaryl, halogen, CF₃, OCF₃, oxo, (OCH₂CH₂O)₁₋₁₀OH, and (OCH₂CH₂O)₁₋₁₀OCH₃;
or an enantiomer, diastereoisomer, racemic mixture, or salt(s) thereof.

21. The method of claim 20, wherein the cell is cancer cell.

22. The method of claim 20 or 21, wherein the cell is in a patient.

23. The method of claim 22, wherein the patient is a cancer patient.

24. The method of any of claims 20-23, wherein the subject has, is suspected of having, or is diagnosed with breast cancer, colon cancer, lung cancer, hepatocellular cancer, pancreatic cancer, prostate cancer, glioblastoma, melanoma, or ovarian cancer.

25. The method of any of claims 20-24, wherein the RAL-interacting protein is RLIP76.

26. The method of any of claims 20-25, wherein the RAL-interacting protein inhibitor binds to a transporter substrate binding site of RLIP76.

27. The method of any of claims 20-26, wherein providing the compound of formula (I) overcomes deleterious effects of p53 gene loss.

28. The method of any of claims 20-25x1, wherein providing the RAL-interacting protein inhibitor to the cell is performed more than once.

29. The method of any of claims 1-28, wherein the compound of formula (I) is provided at a dose between approximately 0.001 μM to 1 mM.

30. The method of any of claims 1-26x1, wherein any hydrogen or carbon present in the compound are replaced by a corresponding isotope ²H, ³H, ¹³C or ¹⁴C.

31. The method of any of claims 20-26x2, wherein the compound of formula (I) is further defined as:

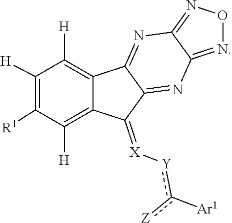

32. The method of any of claims 20-26x1, wherein the RAL-interacting protein inhibitor is at least one of the following compounds

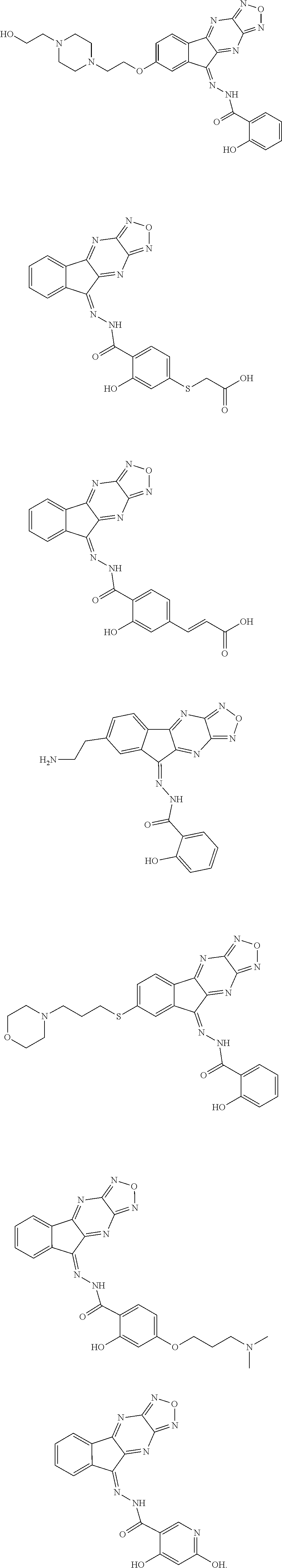

33. The method of any of claims 20-32, wherein the RAL-interacting protein inhibitor is not

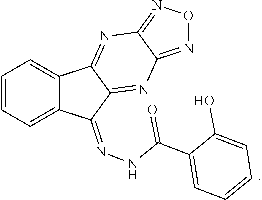 [SMILES: O=C(N/N=C1/c2ccccc2-c2nc3nonc3nc21)c1ccccc1O]

34. A method of treating or preventing cancer in a subject, the method comprising: administering to the subject an effective amount of a compound of formula (II): 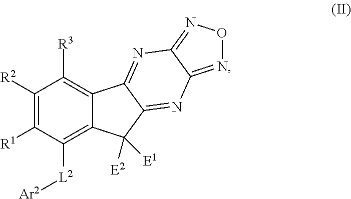
wherein
E¹ and E² are independently H or F, or one of E and E² is H and the other OH, CCH or CN, or E and E² may be taken together to be ═O or ═CH₂;
Ar² is 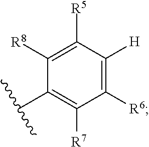
L₂ is —CH₂CH₂—, —CH₂CHR²—, —CH₂NH—, —NHCH₂—, —CH₂NR³—, —NHCHR³—, —CH₂O—, —OCH₂—, —OCHR³—, —COCH₂—, —COCHR²—, —CH₂CO—, —CH₂S—, —CH₂SO—, —CH₂SO(NH)—, —SCH₂—, —SOCH₂—, —SO₂CH₂—, —SO(NH)CH₂—, —SCHR³—, —SOCHR³—, —SO₂CHR³—, —SO(NH)CHR³—, —CH₂SO₂—, —SO₂CHR²—, —SOCHR²—, —SONHCHR²—, —NHS—, —NHSO—, —NHSO₂—, —SNH—, —SONH—, —SO₂NH—, —NHNH—, —SNR³—, —SONR³—, —SO₂NR³—, —NHR³—, —R³NH—, —NHO—, —ONH—, CONH—, —NHCO—, —CONH—, —ONR³—, —NR³CO—, —CONR³—, —OCO— or —COO—;
R¹ is OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, a substituted or unsubstituted heterocycloalkoxy, or a substituted or unsubstituted heterocyclosulfide;
R² is H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
R³ is C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, with the proviso that the L¹ directly bonded to L² is only selected from a bond, CO, CONR⁴ or CO₂;
R⁴ is H or C₁₋₆ lower alkyl;
R⁵ is NH₂, OH, or F;
R⁶ is H, F, Cl, Br, CH₃, ethynyl or cyano;
R⁷ is H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄alkylene-L¹)ₚ;
R⁸ is CH₂OH, CH₂F, CHF₂, CF₃, CHO, CO₂H, CO₂Me, CONH₂, or CONHMe;
R⁹ and R¹⁰ are each independently H, R³, or R⁹ and R¹⁰, and the N atom that connects R⁹ and R¹⁰ can form a heterocyclic ring of 4 to 8 members which can further comprise N, O, S, or SO₂;
R¹¹ is C₁₋₆ lower alkyl, C₂₋₆ lower alkenyl, C₂₋₆ lower alkynyl, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
L¹ is independently at each occurrence a bond, O, NH, NR⁴, N(C₁₋₆ lower acyl), S, SO, SO₂, CO, CONR⁴, CO₂, NR⁴CO, OCONH, OCONR⁴, OCO₂, NR⁴CONR⁴, NR⁴CSNR⁴, NR⁴CO, OCO, SO₂NR⁴, or NR⁴SO; and
p is 1-3, wherein each repeat unit can be the same or different with respect to both cyclic and linear elements;
wherein each of R¹—R¹¹, independently, can optionally be substituted with up to 3 substituents from the following list, OH, CN, C₁₋₆ lower alkyl, C₁₋₆ lower alkoxy, C₁₋₆ lower alkylthio, C₁₋₆ lower alkylamino, C₁₋₆ lower dialkylamino, C₁₋₆ lower acyloxy, optionally substituted phenyl, optionally substituted C₅₋₆ heteroaryl, halogen, CF₃, CF₃O, oxo, (OCH₂CH₂O)₁₋₁₀OH, and (OCH₂CH₂O)₁₋₁₀OCH₃;
or an enantiomer, diastereoisomer, racemic mixture, or salt(s) thereof.

35. The method of claim 34, wherein any hydrogen or carbon present in the compound are replaced by a corresponding isotope ²H, 3H, ¹³C or ¹⁴C.

36. The method of claim 34 or 35, wherein the compound inhibits a RAL-interacting protein.

37. The method of any of claims 34-36, wherein the RAL-interacting protein is RLIP76.

38. The method of any of claims 34-37, wherein the compound binds to a transporter substrate binding site of RLIP76.

39. The method of any of claims 34-38, wherein administration of the compound overcomes deleterious effects of p53 gene loss.

40. The method of any of claims 34-39, wherein the subject has cancer, is suspected of having cancer, or has been diagnosed with cancer.

41. The method of any of claims 34-40, wherein the cancer is breast cancer, colon cancer, lung cancer, hepatocellular cancer, pancreatic cancer, prostate cancer, glioblastoma, melanoma, or ovarian cancer.

42. The method of any of claims 34-41, wherein the breast cancer is triple-negative breast cancer, ductal carcinoma in situ, invasive ductal carcinoma, tubular carcinoma of a breast, medullary carcinoma of a breast, mucinous carcinoma of a breast, papillary carcinoma of a breast, cribriform carcinoma of a breast, invasive lobular carcinoma, inflammatory breast cancer, lobular carcinoma in situ, male breast cancer, molecular subtypes of breast cancer, Paget's disease of a nipple, phyllodes tumors of a breast, metastatic breast cancer, or combinations thereof.

43. The method of any of claims 34-42, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

44. The method of any of claims 34-43, wherein the administration is done prior to, concurrently with, or subsequent to chemotherapy, surgical treatment, or radiation treatment.

45. The method of any of claims 34-44, wherein the compound is administered to the subject at least two, three, four, five, six, seven, eight, nine or ten times.

46. The method of any of claims 34-45, wherein the subject is administered at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 20, 30, 40, 50, 60, 70, 80, 90, 100, 150, 200, 250, or 300 μg/kg or mg/kg.

47. The method of any of claims 34-46, wherein said subject is further administered a distinct cancer therapy.

48. The method of any of claims 34-47, wherein said distinct cancer therapy comprises surgery, radiotherapy, chemotherapy, toxin therapy, immunotherapy, cryotherapy or gene therapy.

49. The method of any of claims 34-48, wherein the compound of formula (II) is further defined as:

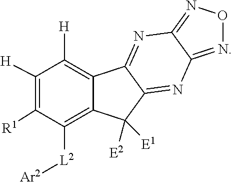

50. A method for inhibiting a RAL-interacting protein in a cell comprising providing to the cell an effective amount of a RAL-interacting protein inhibitor, wherein the RAL-interacting protein inhibitor is a compound of the formula (II): 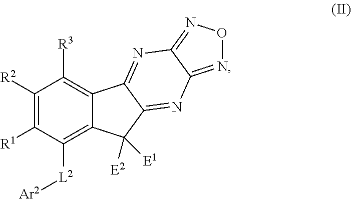
wherein
E¹ and E² are independently H or F, or one of E and E² is H and the other OH, CCH or CN, or E and E² may be taken together to be ═O or ═CH₂;
Ar² is R⁷ 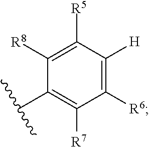
L₂ is —CH₂CH₂—, —CH₂CHR²—, —CH₂NH—, —NHCH₂—, —CH₂NR³—, —NHCHR³—, —CH₂O—, —OCH₂—, —OCHR³—, —COCH₂—, —COCHR²—, —CH₂CO—, —CH₂S—, —CH₂SO—, —CH₂SO(NH)—, —SCH₂—, —SOCH₂—, —SO₂CH₂—, —SO(NH)CH₂—, —SCHR³—, —SOCHR³—, —SO₂CHR³—, —SO(NH)CHR³—, —CH₂SO₂—, —SO₂CHR²—, —SOCHR²—, —SONHCHR²—, —NHS—, —NHSO—, —NHSO₂—, —SNH—, —SONH—, —SO₂NH—, —NHNH—, —SNR³—, —SONR³—, —SO₂NR³—, —NHR³—, —R³NH—, —NHO—, —ONH—, CONH—, —NHCO—, —CONH—, —ONR³—, —NR³CO—, —CONR³—, —OCO— or —COO—;
R¹ is OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, a substituted or unsubstituted heterocycloalkoxy, or a substituted or unsubstituted heterocyclosulfide;
R² is H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
R³ is C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, with the proviso that the L¹ directly bonded to L² is only selected from a bond, CO, CONR⁴ or CO₂;
R⁴ is H or C₁₋₆ lower alkyl;
R⁵ is NH₂, OH or F;
R⁶ is H, F, Cl, Br, CH₃, ethynyl or cyano;
R⁷ is H, OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄alkylene-L¹)ₚ;
R⁸ is CH₂OH, CH₂F, CHF₂, CF₃, CHO, CO₂H, CO₂Me, CONH₂, or CONHMe;
R⁹ and R¹⁰ are each independently H, R³, or R⁹ and R¹⁰, and the N atom that connects R⁹ and R¹⁰ can form a heterocyclic ring of 4 to 8 members which can further comprise N, O, S, or SO₂;
R¹¹ is C₁₋₆ lower alkyl, C₂₋₆ lower alkenyl, C₂₋₆ lower alkynyl, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
L¹ is independently at each occurrence a bond, O, NH, NR⁴, N(C₁₋₆ lower acyl), S, SO, SO₂, CO, CONR⁴, CO₂, NR⁴CO, OCONH, OCONR⁴, OCO₂, NR⁴CONR⁴, NR⁴CSNR⁴, NR⁴CO, OCO, SO₂NR⁴, or NR⁴SO; and
p is 1-3, wherein each repeat unit can be the same or different with respect to both cyclic and linear elements;
wherein each of R¹—R¹¹, independently, can optionally be substituted with up to 3 substituents from the following list, OH, CN, C₁₋₆ lower alkyl, C₁₋₆ lower alkoxy, C₁₋₆ lower alkylthio, C₁₋₆ lower alkylamino, C₁₋₆ lower dialkylamino, C₁₋₆ lower acyloxy, optionally substituted phenyl, optionally substituted C₅₋₆ heteroaryl, halogen, CF₃, CF₃O, oxo, (OCH₂CH₂O)₁₋₁₀OH, and (OCH₂CH₂O)₁₋₁₀OCH₃;
or an enantiomer, diastereoisomer, racemic mixture, or salt(s) thereof.

51. The method of claim 50, wherein the cell is cancer cell.

52. The method of claim 50 or 51, wherein the cell is in a patient.

53. The method of claim 52, wherein the patient is a cancer patient.

54. The method of any of claims 50-53, wherein the subject has, is suspected of having, or is diagnosed with breast cancer, colon cancer, lung cancer, hepatocellular cancer, pancreatic cancer, prostate cancer, glioblastoma, melanoma, or ovarian cancer.

55. The method of any of claims 50-54, wherein the RAL-interacting protein is RLIP76.

56. The method of any of claims 50-55, wherein the RAL-interacting protein inhibitor binds to a transporter substrate binding site of RLIP76.

52x1. The method of any of claims 46-52, wherein providing the compound of formula (I) overcomes deleterious effects of p53 gene loss.

57. The method of any of claims 50-52x1, wherein providing the RAL-interacting protein inhibitor to the cell is performed more than once.

58. The method of any of claims 50-57, wherein the compound of formula (I) is provided at a dose between approximately 0.001 μM to 1 mM.

59. The method of any of claims 50-53x1, wherein any hydrogen or carbon present in the compound are replaced by a corresponding isotope ²H, ³H, ¹³C or ¹⁴C.

60. The method of any of claims 50-53x2, wherein the compound of formula (II) is further defined as:

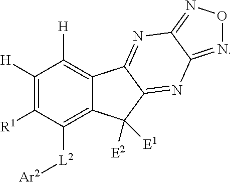

61. A compound of formula (I): 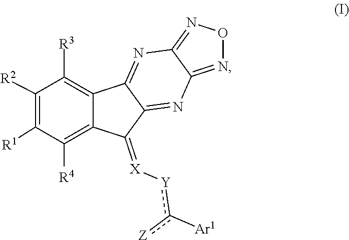
wherein
X is N or CH;
Y is NH or O when Z is a carbonyl oxygen, or CH when Z is F;
Z is ═O or; —F;
R¹ is OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, a substituted or unsubstituted heterocycloalkoxy, or a substituted or unsubstituted heterocyclosulfide,
Ar¹ is 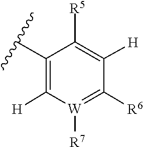
W is N or C;
R² and R⁴ are each independently H, halogen, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkyl-L¹)ₚ, (C₄₋₇ heterocycloalkyl-C₀₋₄ alkylene-L¹)ₚ, (C₆₋₁₀ bridged bicycloheteroalkyl-C₀₋₄ alkylene-L¹)ₚ;
R³ is H, halogen, or C₁₋₆ lower alkyl;
R⁵ is NH₂, OH or F;
R⁶ is H, OH, F, Cl, Br, CH₃, ethynyl, cyano, a substituted or unsubstituted alkenyl, a substituted or unsubstituted alkoxy, or a substituted or unsubstituted sulfide;
R⁷ is nothing when W is N, or H, OH, OR⁹, OCOR¹¹, OCOR⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo,
cyano, nitro, C₁₋₆ lower alkyl, C₂_lower alkenyl, C₂_lower alkynyl, COR¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₄ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ when W is C;
R⁹ and R¹⁰ are each independently H, R³, or R⁹ and R¹⁰, and the N atom that connects R⁹ and R¹⁰ can form a heterocyclic ring of 4 to 8 members which can further comprise N, O, S, or SO₂;
R¹¹ is C₁₋₆ lower alkyl, C₂₋₆ lower alkenyl, C₂₋₆ lower alkynyl, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
L¹ is independently at each occurrence a bond, O, NH, NR⁴, N(C₁₋₆ lower acyl), S, SO, SO₂, CO, CONR⁴, CO₂, NR⁴CO, OCONH, OCONR⁴, OCO₂, NR⁴CONR⁴, NR⁴CSNR⁴, NR⁴CO, OCO, SO₂NR⁴, or NR⁴SO₂;
p is 1-3, wherein each repeat unit can be the same or different with respect to both cyclic and linear elements; and
wherein each of R¹—R¹¹, independently, can optionally be substituted with up to 3 substituents selected from the group consisting of OH, CN, C₁₋₆ lower alkyl, C₁₋₆ lower alkoxy, C₁₋₆ lower alkylthio, C₁₋₆ lower alkylamino, C₁₋₆ lower dialkylamino, C₁₋₆ lower acyloxy, optionally substituted phenyl, optionally substituted C₅₋₆ heteroaryl, halogen, CF₃, OCF₃, oxo, (OCH₂CH₂O)₁₋₁₀OH, and (OCH₂CH₂O)₁₋₁₀OCH₃;
or an enantiomer, diastereoisomer, racemic mixture, or salt(s) thereof.

62. The compound of claim 61, wherein any hydrogen or carbon present in the compound are replaced by a corresponding isotope ²H, ³H, ¹³C or ¹⁴C.

63. The compound of claim 61 or 62, wherein the compound is further defined as:

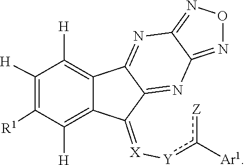

64. The compound of any of claims 61 to 63, wherein the compound is further defined as one of:

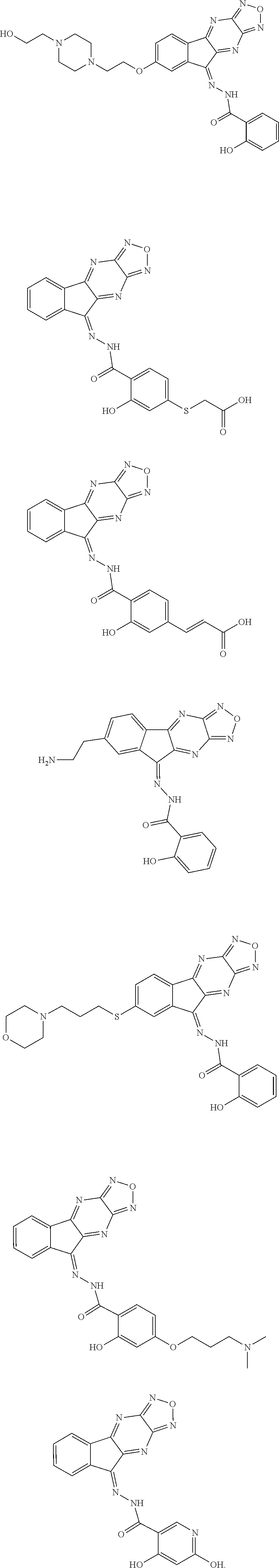

65. A compound of the formula (II): 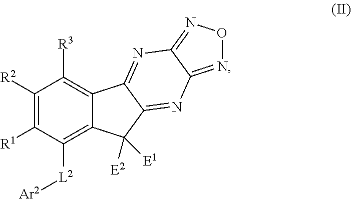
wherein
E¹ and E² are independently H or F, or one of E and E² is H and the other OH, CCH or CN, or E and E² may be taken together to be ═O or ═CH₂;
Ar² is 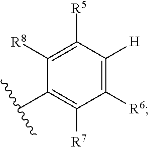
L₂ is —CH₂CH₂—, —CH₂CHR²—, —CH₂NH—, —NHCH₂—, —CH₂NR³—, —NHCHR³—, —CH₂O—, —OCH₂—, —OCHR³—, —COCH₂—, —COCHR²—, —CH₂CO—, —CH₂S—, —CH₂SO—, —CH₂SO(NH)—, —SCH₂—, —SOCH₂—, —SO₂CH₂—, —SO(NH)CH₂—, —SCHR³—, —SOCHR³—, —SO₂CHR³—, —SO(NH)CHR³—, —CH₂SO₂—, —SO₂CHR²—, —SOCHR²—, —SONHCHR²—, —NHS—, —NHSO—, —NHSO₂—, —SNH—, —SONH—, —SO₂NH—, —NHNH—, —SNR³—, —SONR³—, —SO₂NR³—, —NHR³—, —R³NH—, —NHO—, —ONH—, CONH—, —NHCO—, —CONH—, —ONR³—, —NR³CO—, —CONR³—, —OCO— or —COO—;
R¹ is OH, OR⁹, OCOR¹¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₈₋₆ heteroaryl, C₈₋₁₁ bicycloheteroaryl, C₅₋₁₀ bridged bicycloalkyl, C₆-10 bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, a substituted or unsubstituted heterocycloalkoxy, or a substituted or unsubstituted heterocyclosulfide;
R² is H, OH, OR⁹, OCOR¹, OCO₂R⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR¹⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
R³ is C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, (C₆₋₁₀ bridged bicycloheteroalkyl-C₀₋₄ alkylene-L¹)ₚ, with the proviso that the L¹ directly bonded to L² is only selected from a bond, CO, CONR⁴ or CO₂;
R⁴ is H or C₁₋₆ lower alkyl;
R⁵ is NH₂, OH or F;
R⁶ is H, F, Cl, Br, CH₃, ethynyl or cyano;
R⁷ is H, OH, OR⁹, OCOR¹¹, OCOR⁹, OCONHR⁹, OCONR⁹R¹⁰, NH₂ NHR⁹, NR⁹R¹⁰, NHCOR¹¹, NR⁹COR¹¹, NHCO₂R⁹, NR⁹CO₂R⁹, NHCONHR⁹, NR⁹CONR⁹R¹⁰, SR⁹, SOR⁹, SO₂R⁹, NHSO₂R⁹, NR⁰SO₂R⁹, halo, cyano, nitro, C₁₋₆ lower alkyl, C₂₋₆ lower alkenyl, C₂₋₆ lower alkynyl, COR¹¹, CO₂R⁹, CONHR⁹, CONR⁹R¹⁰, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
R⁸ is CH₂OH, CH₂F, CHF₂, CF₃, CHO, CO₂H, CO₂Me, CONH₂, or CONHMe;
R⁹ and R¹⁰ are each independently H, R³, or R⁹ and R¹⁰, and the N atom that connects R⁹ and R¹⁰ can form a heterocyclic ring of 4 to 8 members which can further comprise N, O, S, or SO₂;
R¹¹ is C₁₋₆ lower alkyl, C₂₋₆ lower alkenyl, C₂₋₆ lower alkynyl, C₃₋₆ lower cycloalkyl, C₄₋₆ lower cycloalkenyl, C₄₋₇ lower heterocycloalkyl, phenyl, C₅₋₆ heteroaryl, C₈₋₁₁ bicycloheteroaryl, C₅₋₁₀ bridged bicycloalkyl, C₆₋₁₀ bridged bicycloheteroalkyl, (C₄₋₇ heterocycloalkyl-C₀₋₄ alkylene-L¹)ₚ, or (C₆₋₁₀ bridged bicycloheteroalkyl-C₀₋₄ alkylene-L¹)ₚ;
L¹ is independently at each occurrence a bond, O, NH, NR⁴, N(C₁₋₆ lower acyl), S, SO, SO₂, CO, CONR⁴, CO₂, NR⁴CO, OCONH, OCONR⁴, OCO₂, NR⁴CONR⁴, NR⁴CSNR⁴, NR⁴CO, OCO, SO₂NR⁴, or NR⁴SO; and
p is 1-3, wherein each repeat unit can be the same or different with respect to both cyclic and linear elements;
wherein each of R¹—R¹¹, independently, can optionally be substituted with up to 3 substituents from the following list, OH, CN, C₁₋₆ lower alkyl, C₁₋₆ lower alkoxy, C₁₋₆ lower alkylthio, C₁₋₆ lower alkylamino, C₁₋₆ lower dialkylamino, C₁₋₆ lower acyloxy, optionally substituted phenyl, optionally substituted C₅₋₆ heteroaryl, halogen, CF₃, CF₃O, oxo, (OCH₂CH₂O)₁₋₁₀OH, and (OCH₂CH₂O)₁₋₁₀OCH₃;
or an enantiomer, diastereoisomer, racemic mixture, or salt(s) thereof.

66. The compound of claim 65, wherein any hydrogen or carbon present in the compound are is replaced by a corresponding isotope ²H, ³H, ¹³C or ¹⁴C.

67. The compound of claim 65 or 66, wherein the compound of formula (II) is further defined as: [chemical structure]
